(19) United States
(12) Patent Application Publication
(10) Pub. No.: US 2016/0272970 A1
(43) Pub. Date: Sep. 22, 2016

(54) RNA Interference Agents

(71) Applicant: Arrowhead Madison Inc., Madison, WI (US)

(72) Inventors: David B. Rozema, Cross Plains, WI (US); Darren H. Wakefield, Fitchburg, WI (US); Lauren J. Almeida, Madison, WI (US)

(21) Appl. No.: 15/066,106

(22) Filed: Mar. 10, 2016

Related U.S. Application Data
(60) Provisional application No. 62/134,186, filed on Mar. 17, 2015.
(60) Provisional application No. 62/168,244, filed on May 29, 2015.
(60) Provisional application No. 62/235,816, filed on Oct. 1, 2015.

(51) Int. Cl. C12N 15/113

(52) U.S. Cl. CPC C12N 15/113; C12N 2310/14

(57) ABSTRACT

Described are RNAi interference agents useful in modulating gene expression in a variety of applications, including use in therapeutic, diagnostic, target validation, and genomic discovery applications. Specifically, the invention relates to double stranded modified oligonucleotide molecules having blunt ends and a least one ribonucleotide near the 5′ end of a sense strand capable of mediating RNA interference (RNAi) against target nucleic acid sequences. The RNAi agents are useful in the treatment of diseases or conditions that respond to inhibition of gene expression or activity in a cell, tissue, or organism.

### BACKGROUND

RNA interference (RNAi) is a process by which double-stranded RNA (dsRNA) is used to silence gene expression. The process of post-transcriptional gene silencing is thought to be an evolutionarily-conserved cellular defense mechanism used to prevent the expression of foreign genes. It is currently believed that RNAi begins endogenously with the cleavage of longer dsRNAs into small interfering RNAs (siRNAs) by an RNaseIII-like enzyme, dicer. Dicer-made siRNAs are dsRNAs that are usually about 21-23 nucleotides and often contain 2-nucleotide 3′ overhangs, and 5′ phosphate and 3′ hydroxyl termini. The RNAi response also features an endonuclease complex, commonly referred to as an RNA-induced silencing complex (RISC), which mediates cleavage of single-stranded RNA (mRNA) having sequence complementary to the antisense strand of the siRNA duplex. RISC uses this siRNA strand to identify mRNA molecules that are at least partially complementary to the incorporated siRNA strand, and then cleaves these target mRNAs or inhibits their translation. Cleavage of the target RNA takes place in the middle of the region complementary to the antisense strand of the siRNA duplex (Elbashir et al., 2001, Genes Dev., 15, 188). The siRNA strand that is complementary to the mRNA is known as the guide strand or the antisense strand. The other siRNA strand is known as the passenger strand or the sense strand. Elbashir et al. (Nature 2001) describes RNAi induced by introduction of duplexes of synthetic 21-nucleotide RNAs in cultured mammalian cells. Synthetic siRNA have been subsequently shown to elicit RNA interference in vivo. Examples of RNA-like molecules that can interact with RISC include RNA agents containing one or more chemically modified nucleotides and/or one or more non-phosphodiester linkages.

### SUMMARY

Described herein are RNA interference (RNAi) agents (also RNAi triggers or triggers) comprising: blunt-ended double strand oligonucleotide or RNA-like molecules having a sense strand and an antisense strand wherein the sense strand and the antisense strand are each 26 nucleotides in length (26mers), the antisense strand contains at least 18 consecutive nucleotides that are at least 85% complementary to a sequence in a target mRNA, the sense strand contains at least 18 consecutive nucleotides that are at least 85% complementary to the at least 18 consecutive nucleotides in the antisense strand, and the sense strand further contains at least one ribonucleotide at the second or third position from its 5′ end.

In some embodiments, the antisense strand contains at least 19 consecutive nucleotides that are at least 85% complementary to a sequence in a target mRNA and the sense strand contains at least 19 consecutive nucleotides that are at least 85% complementary to the at least 19 consecutive nucleotides in the antisense strand.

In some embodiments, the antisense strand contains at least 20 consecutive nucleotides that are at least 85% complementary to a sequence in a target mRNA and the sense strand contains at least 20 consecutive nucleotides that are at least 85% complementary to the at least 20 consecutive nucleotides in the antisense strand.

In some embodiments, the antisense strand contains at least 21 consecutive nucleotides that are at least 85% complementary to a sequence in a target mRNA and the sense strand contains at least 21 consecutive nucleotides that are at least 85% complementary to the at least 21 consecutive nucleotides in the antisense strand.

In some embodiments, the antisense strand contains at least 22 consecutive nucleotides that are at least 85% complementary to a sequence in a target mRNA and the sense strand contains at least 22 consecutive nucleotides that are at least 85% complementary to the at least 22 consecutive nucleotides in the antisense strand.

In some embodiments, the antisense strand contains at least 23 consecutive nucleotides that are at least 85% complementary to a sequence in a target mRNA and the sense strand contains at least 23 consecutive nucleotides that are at least 85% complementary to the at least 23 consecutive nucleotides in the antisense strand.

Described herein are RNA interference (RNAi) agents comprising: blunt-ended double strand oligonucleotide or RNA-like molecules having a sense strand and an antisense strand wherein the sense strand and the antisense strand are each 26 nucleotides in length (26mers) and contain a base-paired (complementary) region of at least 18, at least 19, at least 20, at least 21, at least 22, at least 23, at least 24, at least 25, or 26 consecutive nucleotides and the sense strand further contains at least one ribonucleotide at the second or third position from its 5′ end.

The herein described blunt-ended 26mer RNAi agents interact with RISC and participate in RISC-mediated inhibition of gene expression. The herein described blunt-ended 26mer RNAi agents are able to selectively and efficiently decrease expression of a target mRNA.

Described herein are RNAi agents for inhibiting expression of a target gene. The RNAi agent comprises at least two sequences that are at least partially, at least substantially, or fully complementary to each other. The two RNAi agent sequences comprise a sense strand comprising a 26 nucleotide first sequence and an antisense strand comprising a 26 nucleotide second sequence. The RNAi agent sense strands comprise at least 18 consecutive nucleotides that are share at least 85% identity with an at least 18 consecutive nucleotide sequence in a target mRNA. The RNAi agent antisense strands comprise at least 18 consecutive nucleotides that are share at least 85% complementarity with an at least 18 consecutive nucleotide sequence in a target mRNA.

The described RNAi agents can be linked, directly or indirectly, to a targeting group or a delivery polymer. Targeting groups and/or delivery polymers can facilitate delivery of the RNAi agent to a cell in vivo.

The described RNAi agents can be used to provide therapeutic treatments of diseases. Such uses comprise administration of RNAi agent to a human being or animal. For treatment of disease of for formation of a medicament or composition for treatment of a disease, a herein described RNAi agent can be combined with one or more pharmaceutical excipients or with a second therapeutic agent or treatment including, but not limited to: a second RNAi agent or other RNAi agent, a small molecule drug, an antibody or other biologic drug product, an antibody fragment, and/or a vaccine.

The RNAi agents described herein can be delivered to target cells or tissues using any known nucleic acid delivery technology known in the art. Nucleic acid delivery methods include, but are not limited to, encapsulation in liposomes, iontophoresis, or incorporation into other vehicles, such as hydrogels, cyclodextrins, biodegradable nanocapsules, and bioadhesive microspheres, proteinaceous vectors, or DPCs (U.S. Ser. No. 14/452,626 (WO 2015/021092), US-2008-0152661-A1 (WO 2008/0022309), US-2011-0207799-A1 (WO 2011/104169), and WO 2000/053722, each of which is incorporated herein by reference).

The RNAi agents or pharmaceutical compositions containing the RNAi agents described herein can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer: intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration.

### BRIEF DESCRIPTION OF THE DRAWINGS

FIG. 1. Representation of a blunt-ended RNAi agent. N represents a ribonucleotide, deoxyribonucleotide, modified nucleotide, nucleotide mimic, or abasic site. u represents a 2′-O-methyl (2′-OMe) uridine nucleotide. At least one of N^{25′}, N^{24′}, and N^{23′} is a ribonucleic acid. The duplex is substantially complementary (at least 85% complementary between the sense and antisense strand) in the region in which denoted with “|”. “:” represents optional complementarity between the two strands. SS=sense strand. AS=antisense strand.

FIG. 2. Representation of several embodiments (A-C) of blunt-ended RNAi agents. N represents a ribonucleotide, deoxyribonucleotide, modified nucleotide, nucleotide mimic, or abasic site. P represents a ribonucleotide. u represents a 2′-OMe uridine nucleotide. Z represents a 2′-modified nucleotide, a ribonucleotide, or a 2′-deoxyribonucleotide. SS=sense strand. AS=antisense strand. The duplex is substantially complementary (at least 85% complementary between the sense and antisense strand) in the region in which denoted with “|”. “:” represents optional complementarity between the two strands.

FIG. 3. Representation of several embodiments of sense strands (A) or antisense strands (B) of blunt-ended RNAi agents wherein “s” at each location represents an optional phosphorothioate linkage. For each “s” the nucleotide linkage is independently a phosphate or a phosphorothioate linkage. N represents a ribonucleotide, deoxyribonucleotide, modified nucleotide, nucleotide mimic, or abasic site. u represents a 2′-O-methyl (2′-OMe) uridine nucleotide. At least one of N^{25′}, N^{24′}, and N^{23′} is a ribonucleic acid.

FIG. 4. Representation of several embodiments of sense strands (A) or antisense strands (B) of blunt-ended RNAi agents wherein “s” at each location represents a phosphorothioate linkage. N represents a ribonucleotide, deoxyribonucleotide, modified nucleotide, nucleotide mimic, or abasic site. u represents a 2′-O-methyl (2′-OMe) uridine nucleotide. At least one of N^{25′}, N^{24′}, and N_{23′} is a ribonucleic acid.

FIG. 5. Representation of a blunt-ended RNAi agent having A) a frayed end, or B) fully complementary sense and antisense strands. N represents a ribonucleotide, deoxyribonucleotide, modified nucleotide, nucleotide mimic, or abasic site. u represents a 2′-O-methyl (2′-OMe) uridine nucleotide. P represents a ribonucleotide. Z represents a 2′-modified nucleotide, a ribonucleotide, or a 2′-deoxyribonucleotide. The duplex is substantially complementary (at least 85% complementary between the sense and antisense strand) in the region in which denoted with “|”. “:” indicates optional base pairing (i.e. complementarity between the two strands). “x” indicates the nucleotides are not base paired, i.e. they are not complementary. SS=sense strand. AS=antisense strand.

### DETAILED DESCRIPTION

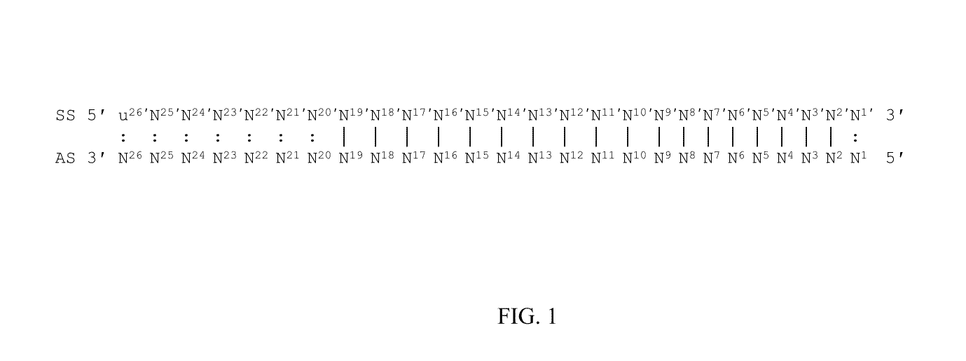

We describe blunt-ended RNAi agents having a sense strand and an antisense strand wherein both the sense strand and the antisense strand are each 26 nucleotides in length. The 26 blunted-ended RNAi agents have the form represented in FIG. 1 wherein at least one or more of positions 23′, 24′, and 25′ is a ribonucleotide, nucleotides at each of positions 23, 24, and 25 is optionally and independently a ribonucleotide, nucleotides at all other positions are modified nucleotides, positions 2-19 are at least 85% complementary to a sequence in a target mRNA, and positions 2′-19′ are at least 85% complementary to corresponding positions 2-19. Unless otherwise noted, when referring to “positions” in the paragraphs that follow, reference to FIG. 1 is envisioned. Nucleotide N¹ is the nucleotide at position 1. Likewise nucleotide N^{1′} is the nucleotide at position 1′. Also with respect to positions, nucleotide N^{26′} is the 5′ terminal nucleotide of the sense strand, nucleotide N^{25′} is the second nucleotide from the 5′ end of the sense strand, etc.

In some embodiments, nucleotides at positions 2-19, 2-20, 2-21, 2-22, 2-23, 1-18, 1-19, 1-20, 1-21, 1-22, or 1-23 are at least 85%, at least 90%, or 100% complementary to a sequence in a target mRNA. In some embodiments, nucleotides at positions 2′-19′, 2′-20′, 2′-21′, 2′-22′, 2′-23′, 1′-18′, 1′-19′, 1′-20′, 1′-21′, 1′-22′, or 1′-23′ are at least 85%, at least 90%, or 100% complementary to the corresponding sequence in the antisense strand.

For the RNAi agents described herein, the following notation is used: N (capital letter without additional notation), unless otherwise indicated, represents a ribonucleotide, deoxyribonucleotide, modified nucleotide, nucleotide mimic, or abasic nucleotide. N can be, but is not limited to, any of the natural or modified nucleotides described herein. P (capital letter) is a ribonucleotide. n (lower case letter) represents a 2′-OMe nucleotide. Nf represents a 2′-fluoro (2′-deoxy-2′-fluoro) nucleotide. dN represents a 2′-deoxy nucleotide. N_{UNA} (or NUNA) represents a 2′,3′-seco nucleotide (unlocked nucleotide). N_{LNA} (or NLNA) represents a locked nucleotide. Nf_{ANA} (or NfANA represents a 2′-F-Arabino nucleotide. NM (or 2′-MOE) represents a 2′-methoxyethyl nucleotide. X represents an abasic ribose. R represents a ribitol. (invN) represents an inverted nucleotide (3′-3′ linked nucleotide). (invdN) represents an inverted deoxyribonucleotide, (invX) represents an inverted abasic nucleotide. (invn) represents an inverted 2′-OMe nucleotide. (invN) can be, but is not limited to: (invdN), (invX), or (invn). s represents a phosphorothioate linked nucleotide. p represents a phosphate. vpdN represents a vinyl phosphonate deoxyribonucleotide. (3′OMen) represents a 3′-OMe nucleotide.

The described RNAi agents contain at least one ribonucleotide in the sense strand. In some embodiments, the ribonucleotide is a ribopurine (A or G). In some embodiments, at least one of the nucleotides at positions 24′ or 25′ is a ribonucleotide or ribopurine and nucleotides at all other positions are modified. In some embodiments, at least one of the nucleotides at positions 24′ or 25′ is a ribonucleotide, at least one of the nucleotides at positions 23, 24 or 25 is a ribonucleotide, and nucleotides at all other positions are modified.

In some embodiments, the nucleotide sequence at positions u^{26′}N^{25′}N^{24′}N^{23′} (5′ end of the sense strand) is selected from the group consisting of: uPuZ, uuPP, uPPu, uAuZ, uGuZ, uuAA, uuGG, uuAG, uuGA, uAAu, uGGu, uAGu, and uGAu, wherein P is a ribonucleotide or a ribopurine and Z is a 2′-modified nucleotide, a ribonucleotide, or a deoxynucleotide.

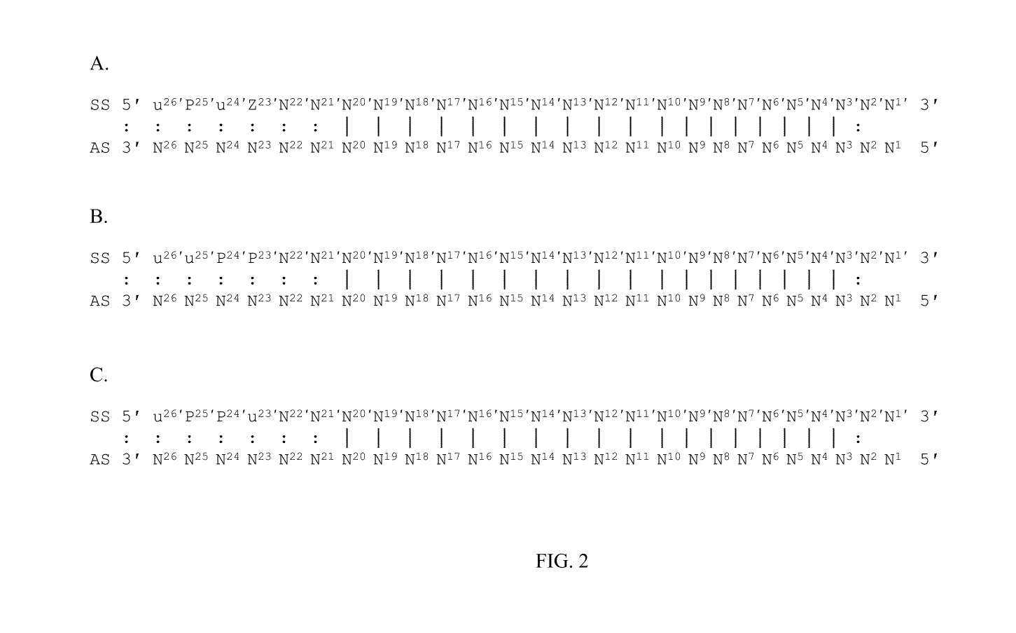

In some embodiments, as represented in FIG. 2, position 26′ is a 2′-OMe uridine. In some embodiments, position 25′ is a ribonucleotide, a ribopurine (2′-OH adenosine (ribo-adenosine) or 2′-OH guanosine (ribo-guanosine)), or a 2′-OMe uridine. In some embodiments, if position 25′ is a ribonucleotide or a ribopurine, position 24′ is 2′-OMe uridine, a ribonucleotide or a ribopurine (2′-OH adenosine (ribo-adenosine) or 2′-OH guanosine (ribo-guanosine)). In some embodiments, if position 25′ is a 2′-OMe uridine, position 24′ is a ribonucleotide or ribopurine (2′-OH adenosine (ribo-adenosine), or 2′-OH guanosine (ribo-guanosine)). In some embodiments, if positions 25′ and 24′ are each a ribonucleotide or ribopurine, position 23′ is 2′-OMe uridine. In some embodiments, position 25′ is a ribonucleotide or a ribopurine and 24′ is a 2′-OMe uridine, position 23′ is a 2′-modified nucleotide, a ribonucleotide, a ribopurine, or a deoxynucleotide. In some embodiments, if position 25′ is a 2′-OMe uridine and 24′ is a ribonucleotide or a ribopurine, position 23′ is a ribonucleotide or a ribopurine.

In some embodiments, positions 26′-24′ are uAu or uGu wherein A and G are ribonucleotides. In some embodiments, positions 26′-24′ are uuA or uuG wherein A and G are ribonucleotides In some embodiments, positions 26′-23′ are uAuA, uAuG, uGuA, uGuG or uNuN wherein A, G, and N are ribonucleotides. In some embodiments, positions 26′-23′ are uuAu, uuGA, or uUaG wherein A, G, and U are ribonucleotides. In some embodiments, positions 26′-22′ are UAUUA wherein U and A are ribonucleotides.

In some embodiments the terminal 3′ nucleotide (N^{1′}) of the sense strand is Nf. In some embodiments the terminal 3′ nucleotide of the sense strand is Af. In some embodiments the terminal 3′ nucleotide of the sense strand is n. In some embodiments the terminal 3′ nucleotide of the sense strand is a. In some embodiments the terminal 3′ nucleotide of the sense strand is c. In some embodiments the terminal 3′ nucleotide of the sense strand is u. In some embodiments the terminal 3′ nucleotide of the sense strand is g. In some embodiments the terminal 3′ nucleotide of the sense strand is u. In some embodiments the terminal 3′ nucleotide of the sense strand is (invN). In some embodiments the terminal 3′ nucleotide of the sense strand is (invdN). In some embodiments the terminal 3′ nucleotide of the sense strand is (inva). In some embodiments the terminal 3′ nucleotide of the sense strand is (3′OMen). In some embodiments the terminal 3′ nucleotide of the sense strand is (3′OMea). In some embodiments the terminal 3′ nucleotide of the sense strand is NM. In some embodiments the terminal 3′ nucleotide of the sense strand is CM.

In some embodiments, the terminal 5′ nucleotide (N¹) of the antisense strand is dN. In some embodiments, the terminal 5′ nucleotide of the antisense strand is dT. In some embodiments, the terminal 5′ nucleotide of the antisense strand is n. In some embodiments, the terminal 5′ nucleotide of the antisense strand is u. In some embodiments, the terminal 5′ nucleotide of the antisense strand is a. In some embodiments, the terminal 5′ nucleotide of the antisense strand is (invN). In some embodiments, the terminal 5′ nucleotide of the antisense strand is (invdN).

In some embodiments, the terminal 5′ nucleotide of the antisense strand is (invdA). In some embodiments, the terminal 5′ nucleotide of the antisense strand is (invAbasic or invX). In some embodiments, the terminal 5′ nucleotide of the antisense strand is (invn). In some embodiments, the terminal 5′ nucleotide of the antisense strand is (invu). In some embodiments, the terminal 5′ nucleotide of the antisense strand is Abasic. In some embodiments, the terminal 5′ nucleotide of the antisense strand is (3′OMen). In some embodiments, the terminal 5′ nucleotide of the antisense strand is NM. In some embodiments, the terminal 5′ nucleotide of the antisense strand is (3′OMeu).

In some embodiments the five nucleotides (5′ N²²-N²⁶ 3′) at the 3′ end of the antisense strand are nnnnn. In some embodiments the five nucleotides at the 3′ end of the antisense strand are nnndNdN. In some embodiments the five nucleotides at the 3′ end of the antisense strand are nnn(invdN)n. In some embodiments the five nucleotides at the 3′ end of the antisense strand are nnnNN. In some embodiments the five nucleotides at the 3′ end of the antisense strand are nnnNn. In some embodiments the five nucleotides at the 3′ end of the antisense strand are nnnNMNM. In some embodiments the five nucleotides at the 3′ end of the antisense strand are nNNNN. In some embodiments the five nucleotides at the 3′ end of the antisense strand are nNnNfn. In some embodiments the five nucleotides at the 3′ end of the antisense strand are nnNfnn. In some embodiments the five nucleotides at the 3′ end of the antisense strand are NfnnNn. In some embodiments the five nucleotides at the 3′ end of the antisense strand are NMNMnNn.

Positions 1 and 1′ are modified nucleotides. In some embodiments, the nucleotide at position 1 is a modified adenosine, modified uridine, or a deoxythimidine. In some embodiments, the nucleotide at position 1′ is a modified adenosine, modified uridine, a deoxythimidine, or an inverted deoxythimidine.

In some embodiments 20% or fewer of the modified nucleotides are 2′-fluoro modified nucleotides.

In some embodiments, the described RNAi agent contains at least one modified backbone. In some embodiments, the modified backbone is a phosphorothioate linkage. In some embodiments, a sense strand of the described RNAi agents contains 1-4 phosphorothioate linkages. In other embodiments, an antisense strand of the described RNAi agents contains 1-4 phosphorothioate linkages. In yet other embodiments, both the sense strand and the antisense strand contain 1-4 phosphorothioate linkages.

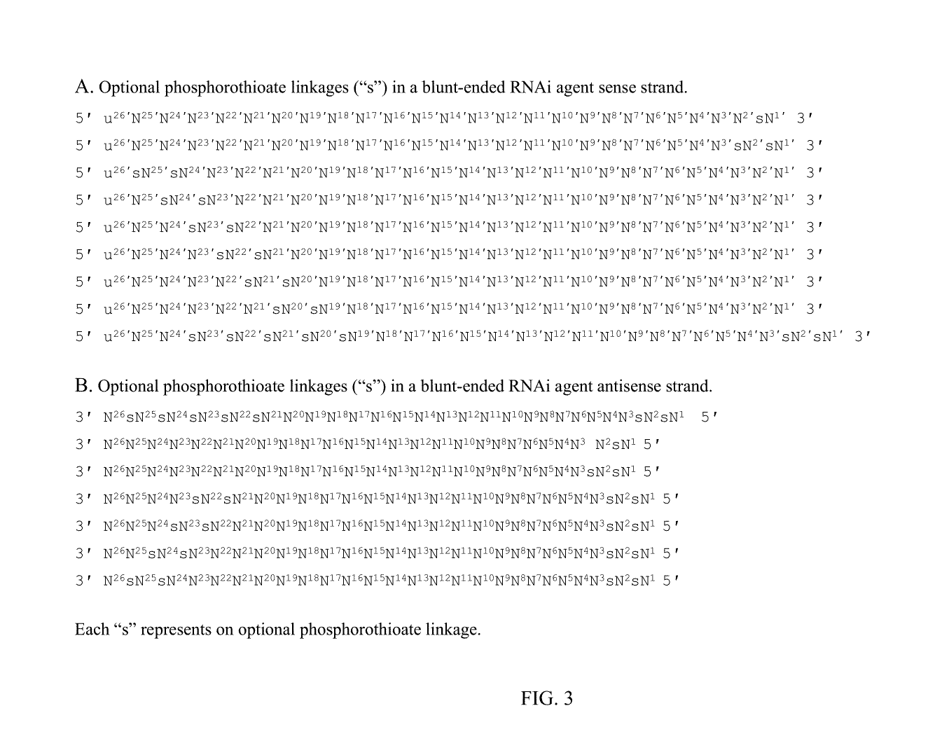

In some embodiments, each of nucleotides 1′-2′, 2′-3′, 1-2, 2-3, 19′-20′, 20′-21′, 21′-22′, 22′-23′, 23′-24′, 21-22, 22-23, 23-24, 24-25, 25-26, is optionally and independently linked via a phosphorothioate linkage (see e.g., FIG. 3).

In some embodiments, the nucleotide at position 1′ is linked to the nucleotide at position 2′ via a phosphorothioate linkage.

In some embodiments, the nucleotide at position 2′ is linked to the nucleotide at position 3′ via a phosphorothioate linkage.

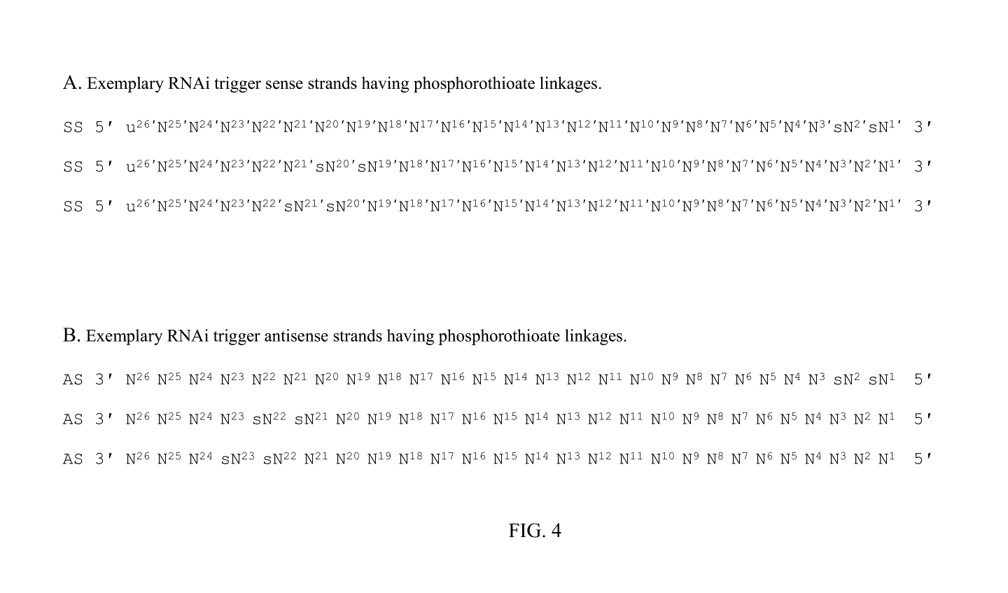

In some embodiments, the nucleotide at position 1′ is linked to the nucleotide at position 2′ via a phosphorothioate linkage and the nucleotide at position 2′ is linked to the nucleotide at position 3′ via a phosphorothioate linkage (FIG. 4A)

In some embodiments, the nucleotide at position 19′ is linked to the nucleotide at position 20′ via a phosphorothioate linkage.

In some embodiments, the nucleotide at position 20′ is linked to the nucleotide at position 21′ via a phosphorothioate linkage.

In some embodiments, the nucleotide at position 19′ is linked to the nucleotide at position 20′ via a phosphorothioate linkage and the nucleotide at position 20′ is linked to the nucleotide at position 21′ via a phosphorothioate linkage. (FIG. 4A)

In some embodiments, the nucleotide at position 20′ is linked to the nucleotide at position 21′ via a phosphorothioate linkage.

In some embodiments, the nucleotide at position 21′ is linked to the nucleotide at position 22′ via a phosphorothioate linkage.

In some embodiments, the nucleotide at position 20′ is linked to the nucleotide at position 21′ via a phosphorothioate linkage and the nucleotide at position 21′ is linked to the nucleotide at position 22′ via a phosphorothioate linkage (FIG. 4A).

In some embodiments, the nucleotide at position 1 is linked to the nucleotide at position 2 via a phosphorothioate linkage.

In some embodiments, the nucleotide at position 2 is linked to the nucleotide at position 3 via a phosphorothioate linkage.

In some embodiments, the nucleotide at position 1 is linked to the nucleotide at position 2 via a phosphorothioate linkage and the nucleotide at position 2 is linked to the nucleotide at position 3 via a phosphorothioate linkage (FIG. 4B).

In some embodiments, the nucleotide at position 21 is linked to the nucleotide at position 22 via a phosphorothioate linkage.

In some embodiments, the nucleotide at position 22 is linked to the nucleotide at position 23 via a phosphorothioate linkage.

In some embodiments, the nucleotide at position 21 is linked to the nucleotide at position 22 via a phosphorothioate linkage and the nucleotide at position 22 is linked to the nucleotide at position 23 via a phosphorothioate linkage (FIG. 4B).

In some embodiments, the nucleotide at position 22 is linked to the nucleotide at position 23 via a phosphorothioate linkage.

In some embodiments, the nucleotide at position 23 is linked to the nucleotide at position 24 via a phosphorothioate linkage.

In some embodiments, the nucleotide at position 22 is linked to the nucleotide at position 23 via a phosphorothioate linkage and the nucleotide at position 23 is linked to the nucleotide at position 24 via a phosphorothioate linkage. (FIG. 4B).

In some embodiments, the nucleotide at position 20′ is linked to the nucleotide at position 21′ via a phosphorothioate linkage, the nucleotide at position 21′ is linked to the nucleotide at position 22′ via a phosphorothioate linkage, the nucleotide at position 22 is linked to the nucleotide at position 23 via a phosphorothioate linkage and the nucleotide at position 23 is linked to the nucleotide at position 24 via a phosphorothioate linkage.

In some embodiments, the nucleotide at position 19′ is linked to the nucleotide at position 20′ via a phosphorothioate linkage, the nucleotide at position 20′ is linked to the nucleotide at position 21′ via a phosphorothioate linkage, the nucleotide at position 21 is linked to the nucleotide at position 22 via a phosphorothioate linkage and the nucleotide at position 22 is linked to the nucleotide at position 23 via a phosphorothioate linkage.

In some embodiments, the nucleotide at position 22′ is linked to the nucleotide at position 23′ via a phosphorothioate linkage.

In some embodiments, the nucleotide at position 23′ is linked to the nucleotide at position 24′ via a phosphorothioate linkage.

In some embodiments, the nucleotide at position 23 is linked to the nucleotide at position 24 via a phosphorothioate linkage.

In some embodiments, the nucleotide at position 24 is linked to the nucleotide at position 25 via a phosphorothioate linkage.

In some embodiments, the nucleotide at position 25 is linked to the nucleotide at position 26 via a phosphorothioate linkage.

As used herein, the term “sequence” or “nucleotide sequence” refers to a succession or order of nucleobases or nucleotides, described with a succession of letters using the standard nucleotide nomenclature and the key for modified nucleotides described herein.

As used herein, and unless otherwise indicated, the term “complementary,” when used to describe a first nucleotide sequence (e.g. RNAi agent sense strand or target mRNA) in relation to a second nucleotide sequence (e.g. RNAi agent antisense strand), refers to the ability of an oligonucleotide or polynucleotide comprising the first nucleotide sequence to hybridize (form base pair hydrogen bonds) and form a duplex or double helical structure under certain conditions with an oligonucleotide or polynucleotide comprising the second nucleotide sequence. Complementary sequences include Watson-Crick base pairs or non-Watson-Crick base pairs and include natural or modified nucleotides or nucleotide mimics, at least to the extent that the above requirements with respect to the ability to hybridize are fulfilled. Perfectly or fully complementary means that all (100%) of the bases in a contiguous sequence of a first polynucleotide will hybridize with the same number of bases in a contiguous sequence of a second polynucleotide. The contiguous sequence may comprise all or a part of a first or second nucleotide sequence. As used herein, partial complementary means that in a hybridized pair of nucleobase sequences, at least 70% of the bases in a contiguous sequence of a first polynucleotide will hybridize with the same number of bases in a contiguous sequence of a second polynucleotide. As used herein, substantial complementary means that in a hybridized pair of nucleobase sequences, at least 85% of the bases in a contiguous sequence of a first polynucleotide will hybridize with the same number of bases in a contiguous sequence of a second polynucleotide. The terms “complementary”, “fully complementary” and “substantially complementary” herein may be used with respect to the base matching between the sense strand and the antisense strand of an RNAi agent, or between the antisense strand of a RNAi agent and a sequence of a target mRNA.

Sequence identity or complementarity is independent of modification. For example, a and Af are complementary to U (or T) and identical to A for the purposes of determining identity or complementarity.

The nucleic acid sequence of positions 2-19 is at least 85% complementary to a nucleotide sequence in a target mRNA. In some embodiments, the nucleic acid sequence of positions 2-19 is at least 90% complementary to a nucleotide sequence in a target mRNA. In some embodiments, the nucleic acid sequence of positions 2-19 is 100% complementary to a nucleotide sequence in a target mRNA.

The nucleic acid sequence of positions 2′-19′ is at least 85% complementary to the corresponding nucleic acid sequence of positions 2-19 or identical to a nucleotide sequence in a target mRNA. In some embodiments, the nucleic acid sequence of positions 2′-19′ is at least 90% complementary to the corresponding nucleic acid sequence of positions 2-19 or identical to a nucleotide sequence in a target mRNA. In some embodiments, the nucleic acid sequence of positions 2′-19′ is 100% complementary to the corresponding nucleic acid sequence of positions 2-19 or identical to a nucleotide sequence in a target mRNA.

Nucleotides N²⁰, N²¹, N²², and N²³ (i.e. nucleotides at positions 20, 21, 22, and 23) are independently and optionally complementary to a corresponding sequence in a target mRNA. In some embodiments, the nucleotide sequence of positions 2-20, 2-21, 2-22, or 2-23 is at least 80%, at least 85%, at least 90%, or 100% complementary to a nucleotide sequence in a target mRNA.

Nucleotides N^{20′} and N^{21′} (i.e. nucleotides at positions 20′ and 21′) are independently and optionally identical to a corresponding sequence in a target mRNA. In some embodiments, the nucleotide sequence of positions 2′-20′ or 2′-21′ is at least 80%, at least 85%, at least 90%, or 100% identical to a nucleotide sequence in a target mRNA.

The nucleotide at position 20 is optionally complementary to the nucleotide at position 20′. The nucleotide at position 21 is optionally complementary to the nucleotide at position 21′. The nucleotide at position 22 is optionally complementary to the nucleotide at position 22′. The nucleotide at position 23 is optionally complementary to the nucleotide at position 23′. The nucleotide at position 24 is optionally complementary to the nucleotide at position 24′. The nucleotide at position 25 is optionally complementary to the nucleotide at position 25′. The nucleotide at position 26 is optionally complementary to the nucleotide at position 26′.

In some embodiments, the nucleotide at position 20 is complementary to the nucleotide at position 20′. In some embodiments, nucleotide at position 21 is complementary to the nucleotide at position 21′. In some embodiments, the nucleotide at position 22 is complementary to the nucleotide at position 22′. In some embodiments, the nucleotide at position 23 is complementary to the nucleotide at position 23′. In some embodiments, the nucleotide at position 24 is complementary to the nucleotide at position 24′. In some embodiments, the nucleotide at position 25 is complementary to the nucleotide at position 25′. In some embodiments, the nucleotide at position 26 is complementary to the nucleotide at position 26′.

In some embodiments, the nucleotide at position 20 is not complementary to the nucleotide at position 20′. In some embodiments, the nucleotide at position 21 is not complementary to the nucleotide at position 21′. In some embodiments, the nucleotide at position 22 is not complementary to the nucleotide at position 22′. In some embodiments, the nucleotide at position 23 is not complementary to the nucleotide at position 23′. In some embodiments, the nucleotide at position 24 is not complementary to the nucleotide at position 24′. In some embodiments, the nucleotide at position 25 is not complementary to the nucleotide at position 25′. In some embodiments, the nucleotide at position 26 is not complementary to the nucleotide at position 26′.

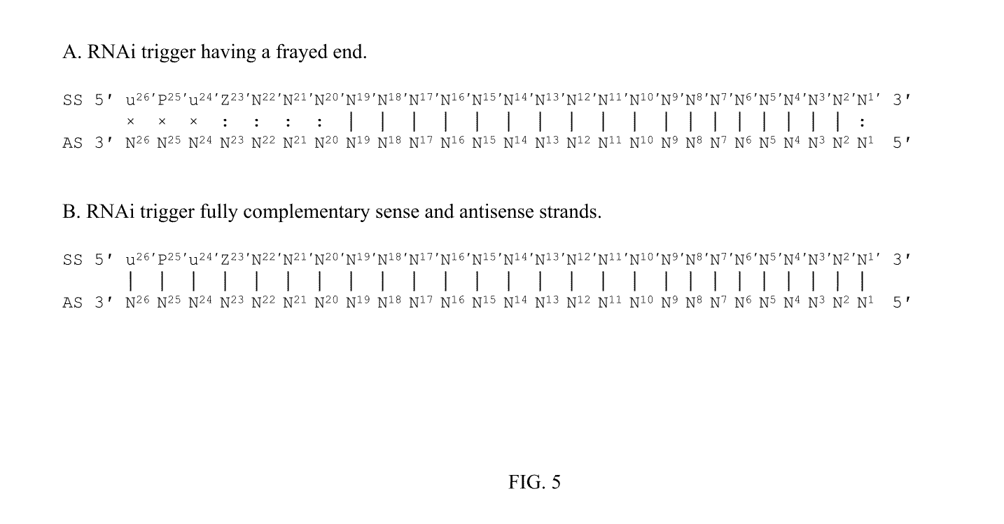

In some embodiments, the nucleotides at positions 25 and 26 are not complementary to the nucleotides at position 25′ and 26′. In some embodiments, the nucleotides at positions 25 and 26 are complementary to the nucleotides at positions 25′ and 26′. In some embodiments, the nucleotides at positions 24, 25, and 26 are not complementary to the nucleotides at position 24′, 25′, and 26′ (as represented in FIG. 5A). In some embodiments, the nucleotides at positions 24, 25, and 26 are complementary to the nucleotides at positions 24′, 25′, and 26′. In some embodiments, the nucleotides at positions 24 and 26 are not complementary to the nucleotides at position 24′ and 26′. In some embodiments, the nucleotides at positions 24 and 26 are complementary to the nucleotides at position 24′ and 26′. In some embodiments, the nucleotides at positions 23, 24, 25, and 26 are not complementary to the nucleotides at position 23′, 24′, 25′, and 26′. In some embodiments, the nucleotides at positions 22, 23, 24, 25, and 26 are complementary to the nucleotides at position 22′, 23′, 24′, 25′, and 26′.

The nucleotide at position 1′ is optionally identical to a corresponding nucleotide in a target mRNA. In some embodiments, the nucleotide at position 1′ is identical to a corresponding nucleotide in a target mRNA. In some embodiments, the nucleotide at position 1′ is not identical to a corresponding nucleotide in a target mRNA.

The nucleotide at position 1 is optionally complementary to a corresponding nucleotide in a target mRNA. In some embodiments, the nucleotide at position 1 is complementary to a corresponding nucleotide in a target mRNA. In some embodiments, the nucleotide at position 1 is not complementary to a corresponding nucleotide in a target mRNA.

In some embodiments, the nucleotide at position 1′ is complementary to the nucleotide at position 1. In some embodiments, the nucleotide at position 1′ is not complementary to the nucleotide at position 1.

In some embodiments, the nucleotide at position 1 is complementary to the nucleotide at position 1′ and to a corresponding nucleotide in a target mRNA. In some embodiments, the nucleotide at position 1 is complementary to the nucleotide at position 1′ and not complementary to a corresponding nucleotide in a target mRNA. In some embodiments, the nucleotide at position 1 is complementary to a corresponding nucleotide in a target mRNA and not complementary to the nucleotide at position 1′. In some embodiments, the nucleotide at position 1 is not complementary to either a corresponding nucleotide in a target mRNA or the nucleotide at position 1′.

In some embodiments, the nucleotide at position 1′ is complementary to the nucleotide at position 1 and identical to a corresponding nucleotide in a target mRNA. In some embodiments, the nucleotide at position 1′ is complementary to the nucleotide at position 1 and not identical to a corresponding nucleotide in a target mRNA. In some embodiments, the nucleotide at position 1′ is identical to a corresponding nucleotide in a target mRNA and not complementary to the nucleotide at position 1. In some embodiments, the nucleotide at position 1′ is not identical to a corresponding nucleotide in a target mRNA and not complementary to the nucleotide at position 1.

In some embodiments, the nucleotide sequence of positions 1-19, 1-20, 1-21, 1-22, or 1-23 is at least 80%, at least 85%, at least 90%, or 100% complementary to a nucleotide sequence in a target mRNA.

In some embodiments, the nucleotide sequence of positions 1′-19′, 1′-20′ or 1′-21′ is at least 80%, at least 85%, at least 90%, at least 95%, or 100% identical to a nucleotide sequence in a target mRNA.

The sense strand and antisense strands of the described RNAi agents are at least partially complementary to each other. In some embodiments the sense strand is at least 70% complementary to the antisense strand. In some embodiments the sense strand is at least 75% complementary to the antisense strand. In some embodiments the sense strand is at least 80% complementary to the antisense strand. In some embodiments the sense strand is at least 84% complementary to the antisense strand. In some embodiments the sense strand is at least 87% complementary to the antisense strand. In some embodiments the sense strand is at least 90% complementary to the antisense strand. In some embodiments the sense strand is at least 95% complementary to the antisense strand. In some embodiments the sense strand is at perfectly complementary to the antisense strand.

An RNAi agent can contain a non-nucleotide group attached to the 3′ or 5′ end of either the sense strand or the antisense strand. In some embodiments, a targeting group, linking group, or delivery vehicle is covalently linked to the sense strand. In some embodiments, the targeting group, linking group, and/or delivery vehicle is linked to the 3′ end (position 1′) and/or the 5′ end (position 26′) of the sense strand. The targeting group, linking group, and/or delivery vehicle is linked directly or indirectly via a linker to the 3′ or 5′ end of the sense strand. In some embodiments, position 1′ is covalently attached, either directly or indirectly via a linker, to a targeting group. In some embodiments, position 26′ is covalently attached, either directly or indirectly via a linker, to a targeting group. In some embodiments, a targeting group is linked to the RNAi agent via a labile, cleavable, or reversible bond or linker/spacer.

A targeting group enhances the pharmacokinetic or biodistribution properties of a molecule to which they are attached to improve cell- or tissue-specific distribution and cell-specific uptake of the conjugate. Binding of a targeting group to a cell or cell receptor may initiate endocytosis. Targeting groups may be monovalent, divalent, trivalent, tetravalent, or have higher valency. Targeting groups can be, but are not limited to, compounds with affinity to cell surface molecule, cell receptor ligands, antibodies, monoclonal antibodies, antibody fragments, and antibody mimics with affinity to cell surface molecules, hydrophobic groups, cholesterol, cholesteryl groups, or steroids. In some embodiments, a targeting group comprises a cell receptor ligand. A variety of targeting groups have been used to target drugs and genes to cells and to specific cellular receptors. Cell receptor ligands may be, but are not limited to: carbohydrates, glycans, saccharides (including, but not limited to: galactose, galactose derivatives (such as N-acetyl-galactosamine), mannose, and mannose derivatives), haptens, vitamins, folate, biotin, aptamers, and peptides (including, but not limited to: RGD-containing peptides, insulin, EGF, and transferrin).

In some embodiments, an RNAi agent as described herein comprises a linking group conjugated to the RNAi agent. The linking group facilitates covalent linkage of the agent to a targeting group or delivery polymer. The linking group may be linked to the 3′ or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments a linking group is conjugated to the 5′ end of an RNAi agent sense strand. Exemplary linking groups, include, but are not limited to: Alk-SMPT-C6, Alk-SS-C6, DBCO-TEG, Me-Alk-SS-C6, and C6-SS-Alk-Me.

A linker or linking group is a connection between two atoms that links one chemical group (such as an RNAi agent) or segment of interest to another chemical group (such as a targeting group or delivery polymer) or segment of interest via one or more covalent bonds. A labile linkage contains a labile bond. A linkage may optionally include a spacer that increases the distance between the two joined atoms. A spacer may further add flexibility and/or length to the linkage. Spacers may include, but are not be limited to, alkyl groups, alkenyl groups, alkynyl groups, aryl groups, aralkyl groups, aralkenyl groups, aralkynyl groups; each of which can contain one or more heteroatoms, heterocycles, amino acids, nucleotides, and saccharides. Spacer groups are well known in the art and the preceding list is not meant to limit the scope of the invention.

Targeting groups and linking groups include, but are not limited to, the compounds represented by the structures below. In some of the targeting group and linking group structures shown below, the structure includes the RNAi agent, denoted by Trigger, RNA, R, or R1 or R2 (i.e. Trigger, RNA or R1 or R2 each comprises the RNAi agent). In some embodiments, the RNAi agent is linked directly to a targeting group or linking group. In other embodiments, the RNAi agent is linked to a targeting group and linking group via a linker. For (Alk-C6-Ser), (Alk-PEGS-Ser), and (Alk-PEG13-Ser), one of R1 and R2 comprises the RNAi agent and the other is a hydrogen. For linkers (C3), (C12), (Sp9), (Sp18), (Spermine), (C6-SS-C6), one of R1 or R2 comprises the RNAi agent and the other comprises a hydrogen, reactive group, targeting group, linking group, alkyl group, or substituted alkyl group.

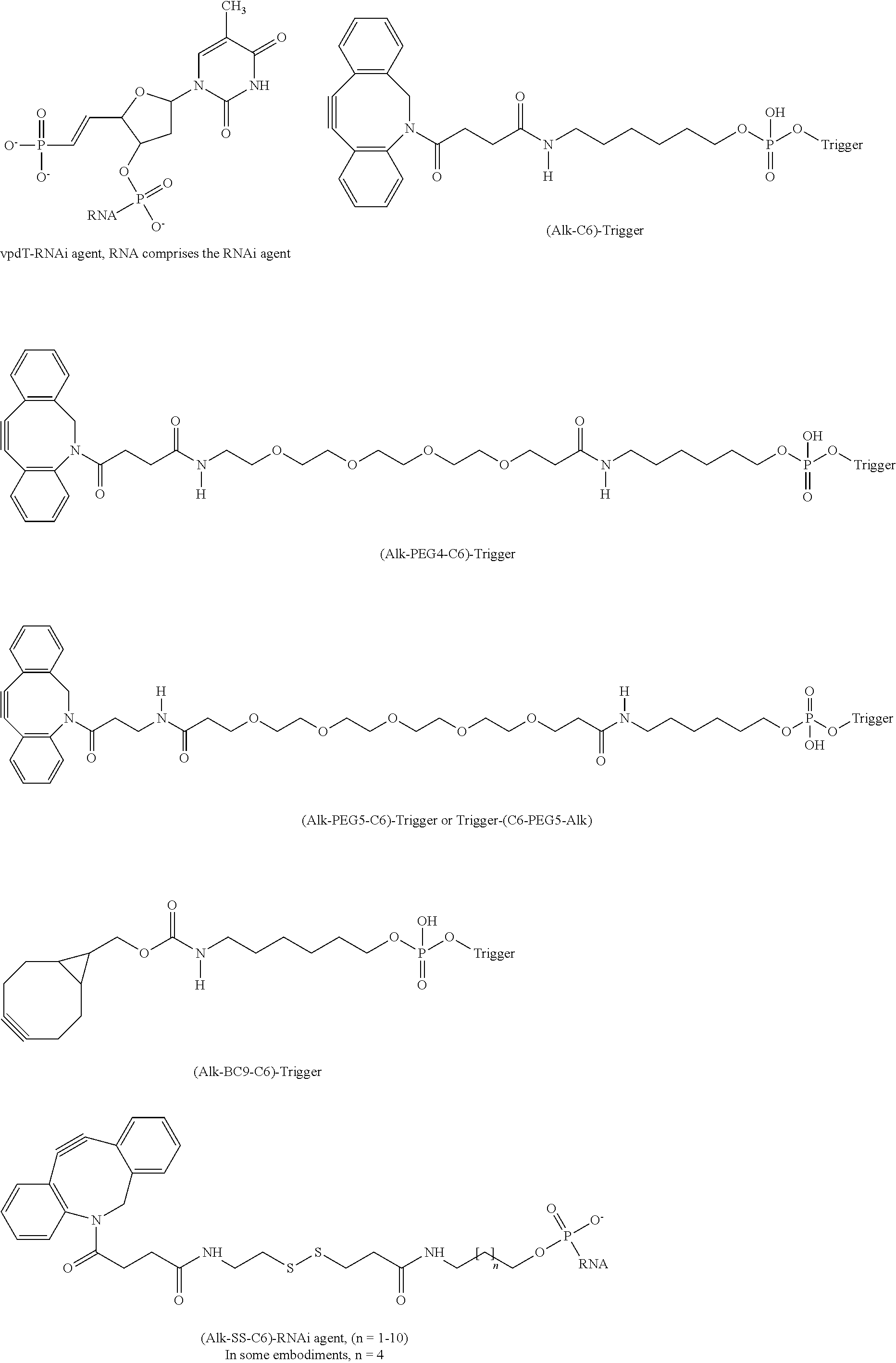

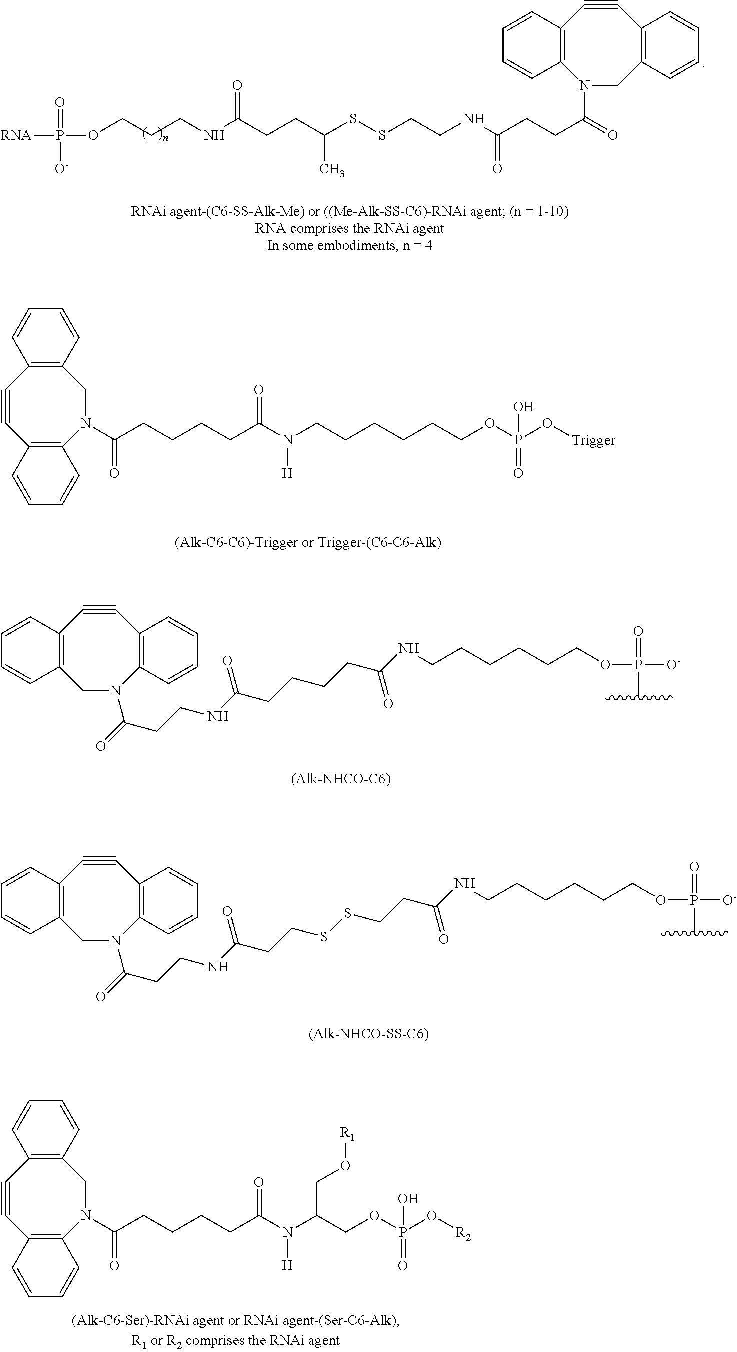

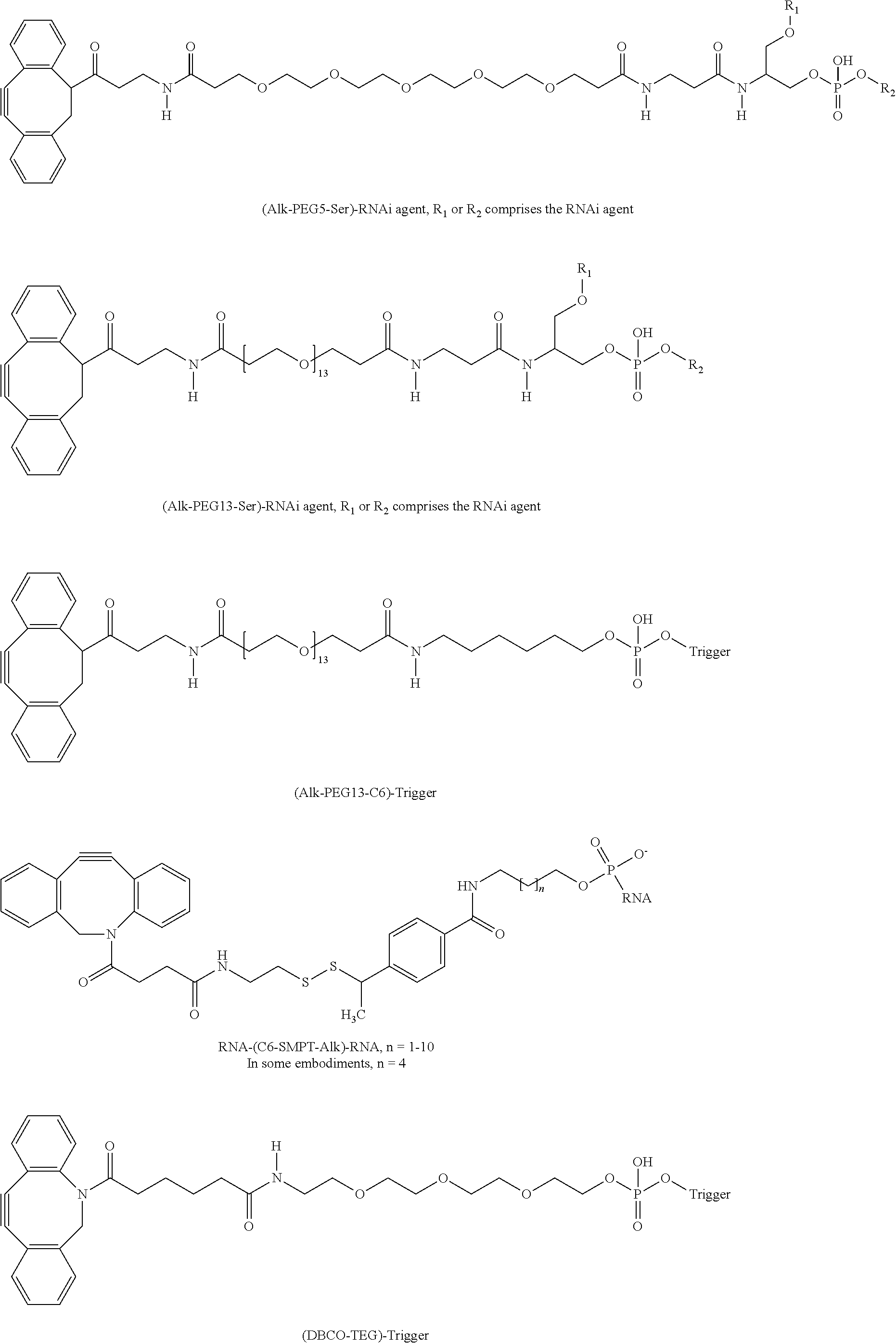

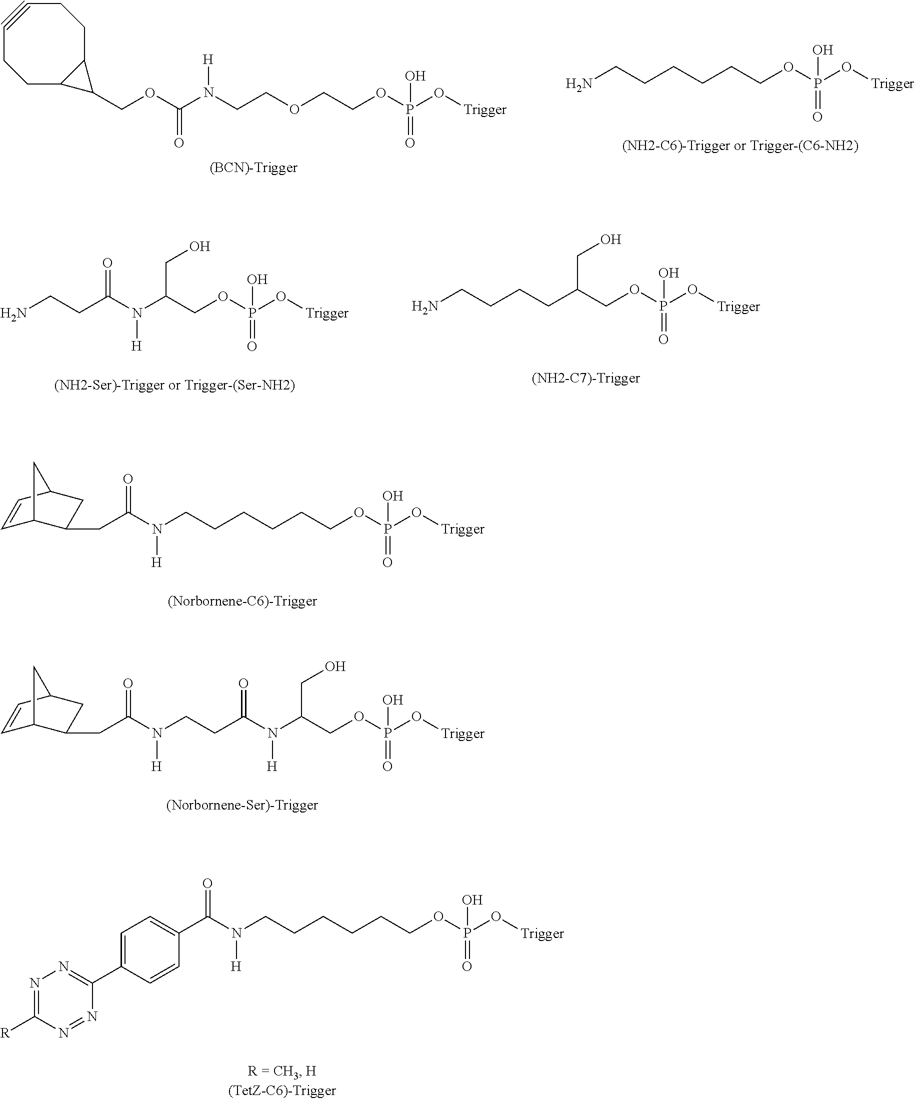

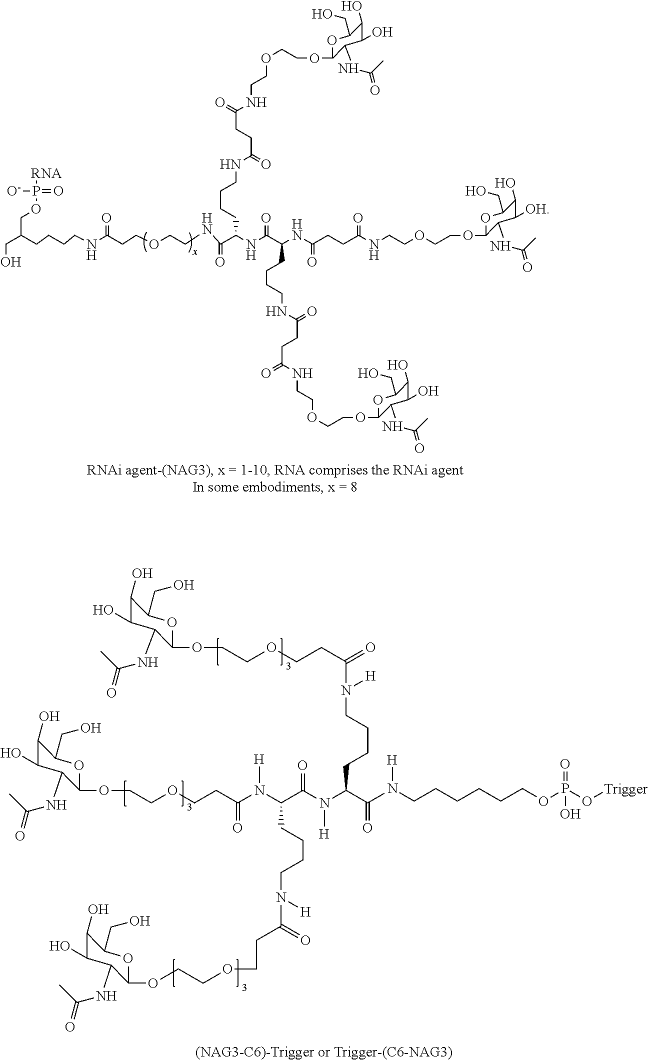

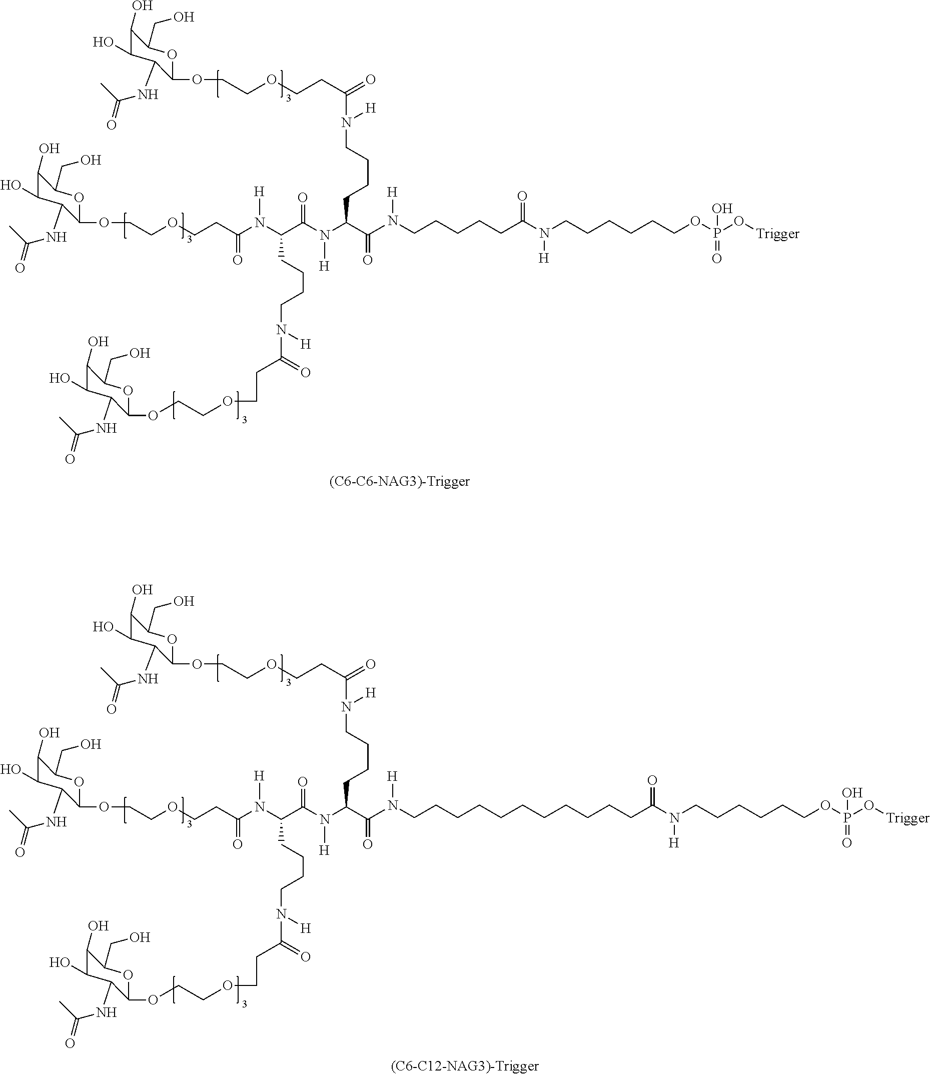

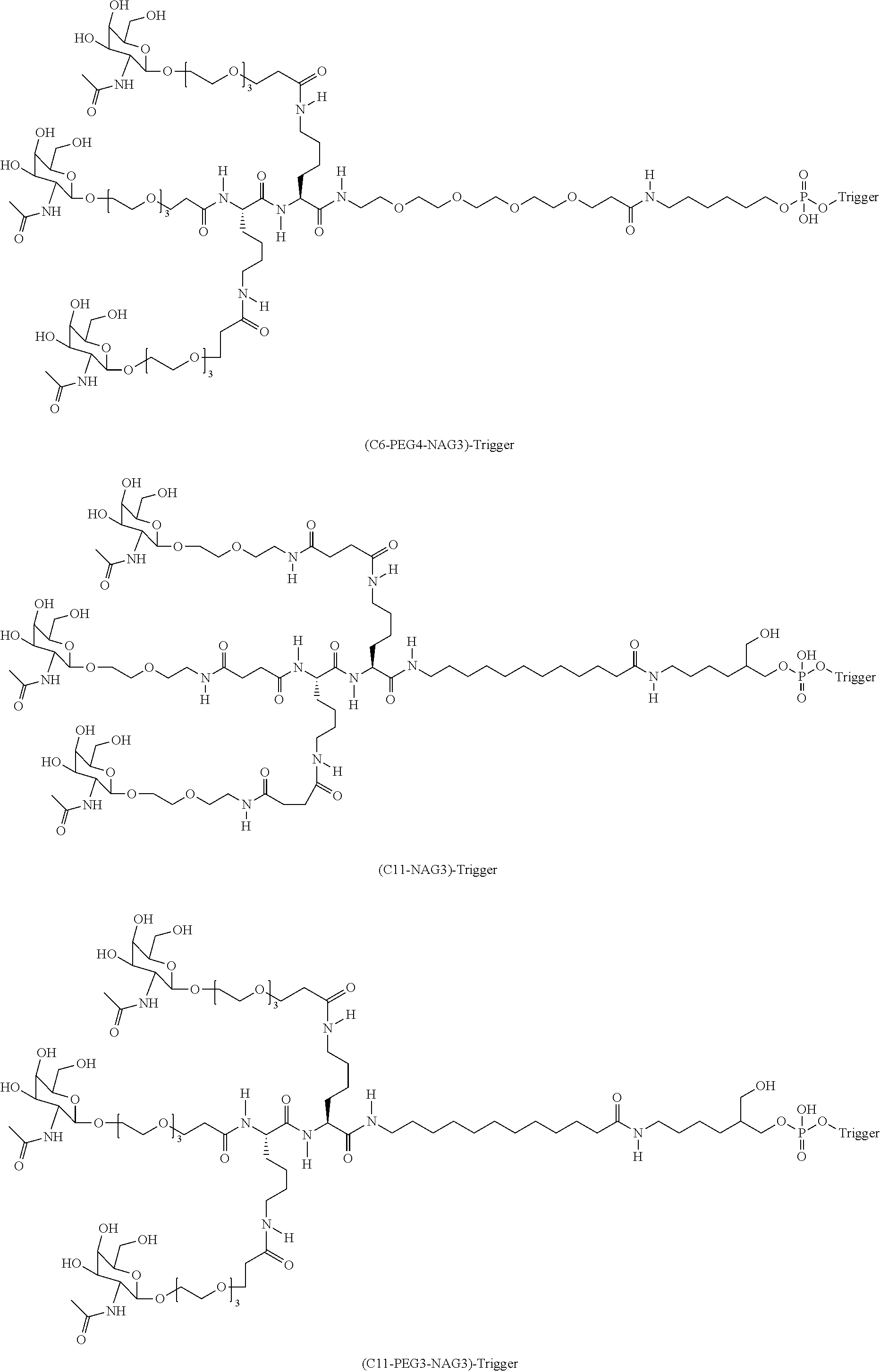

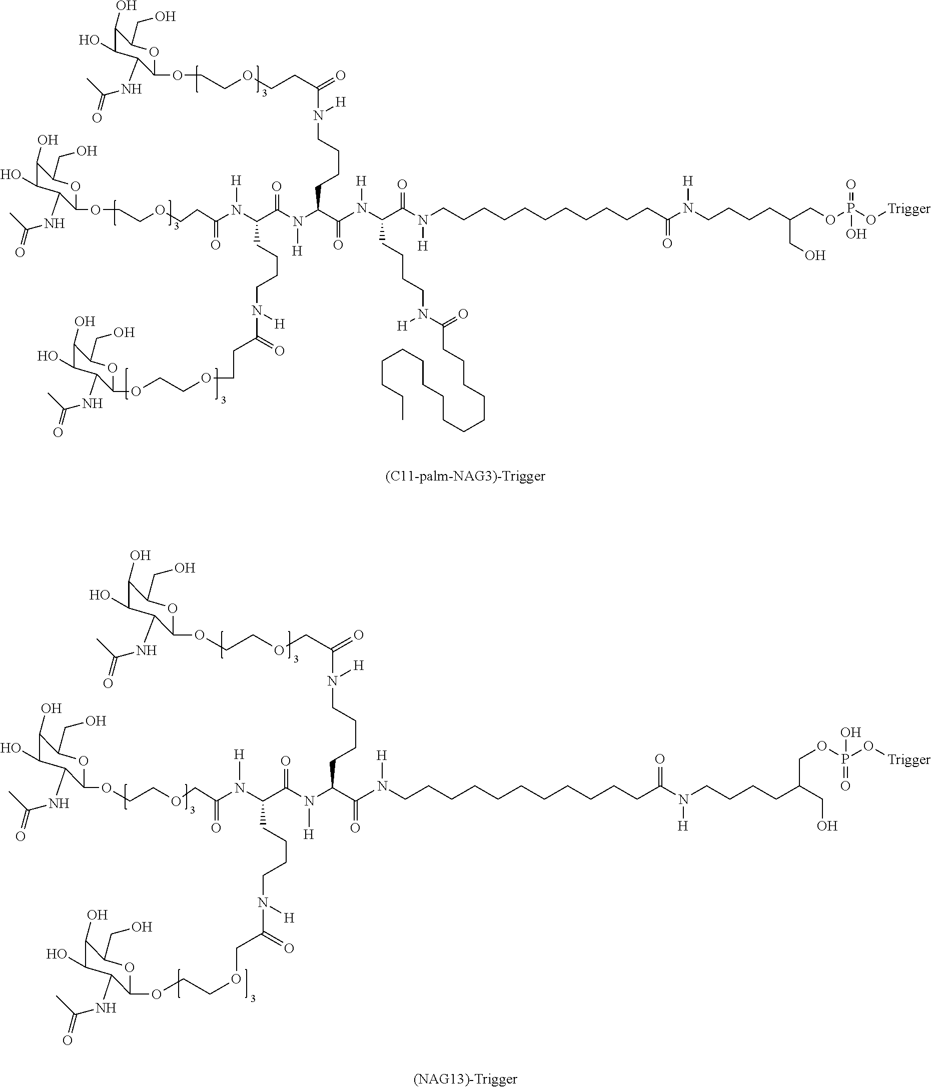

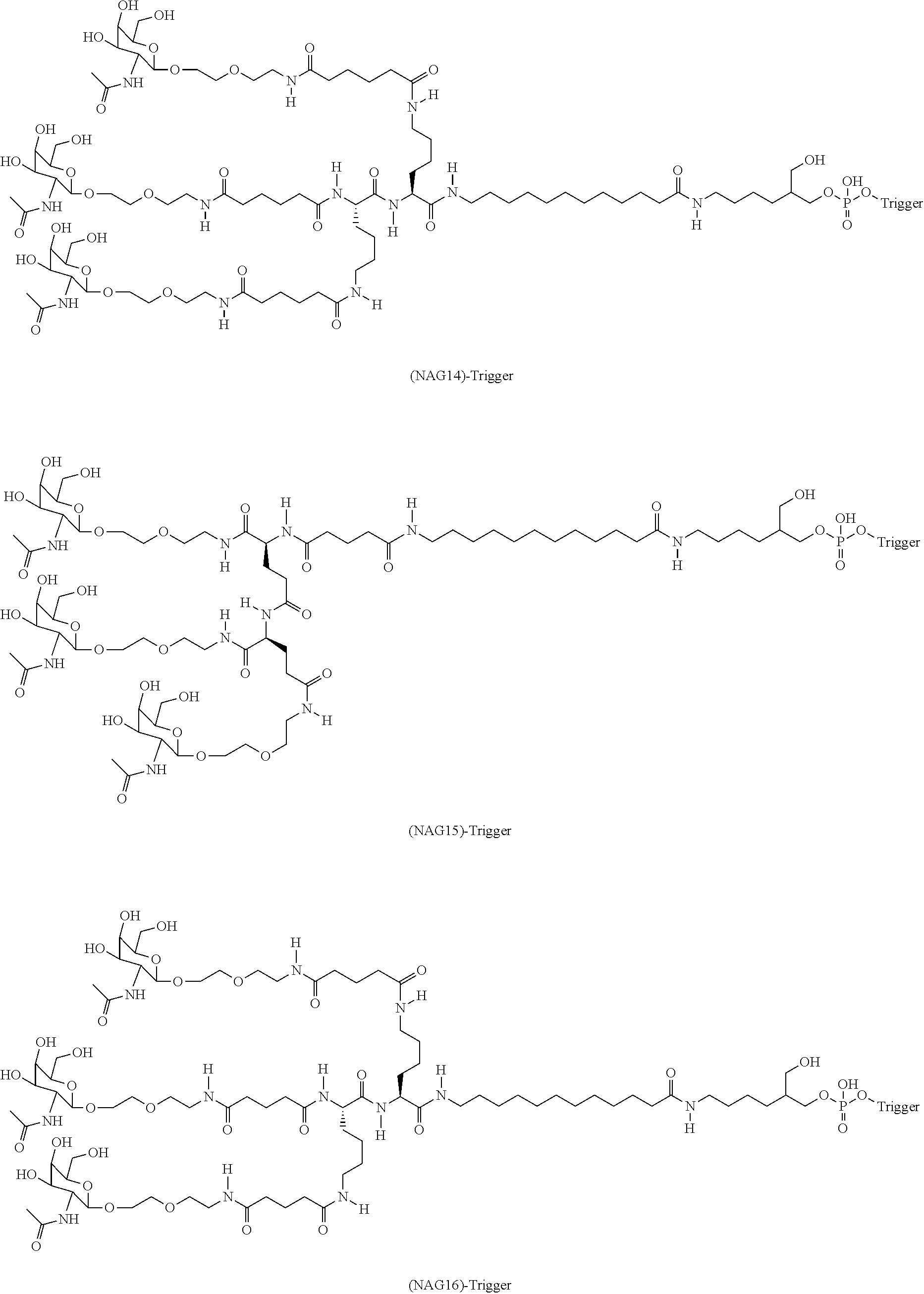

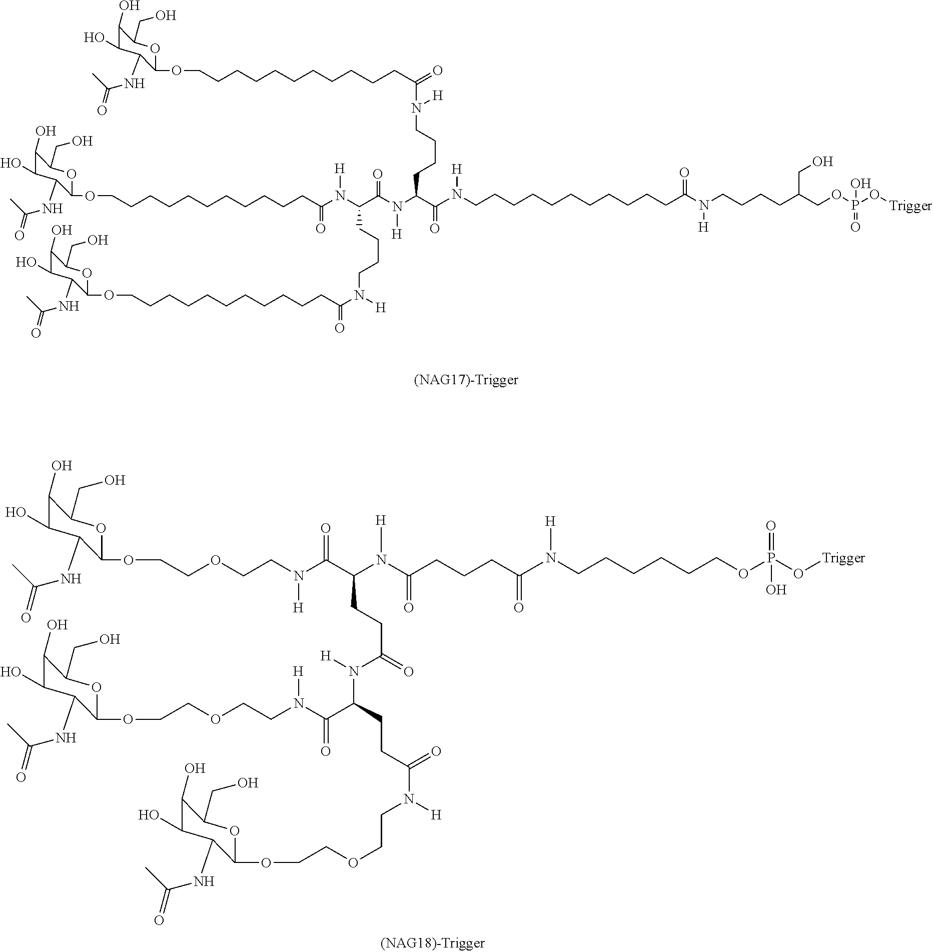

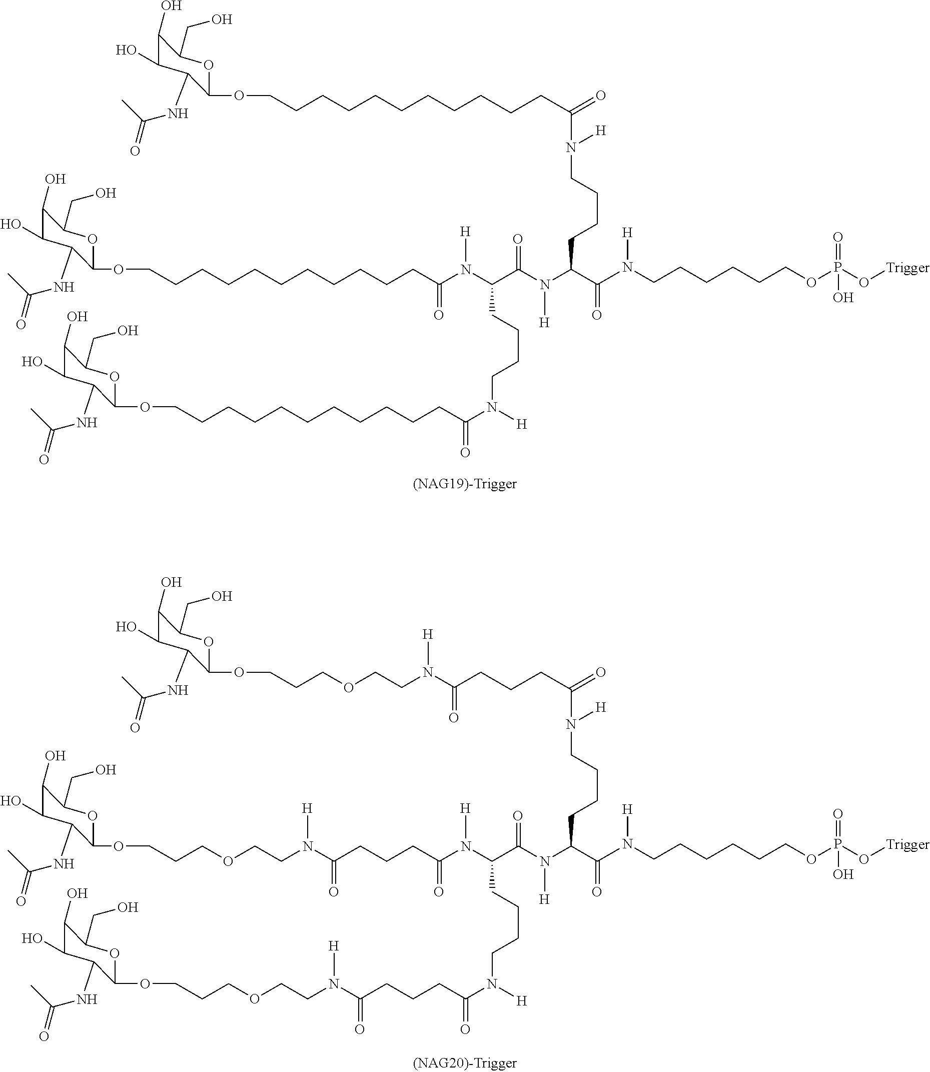

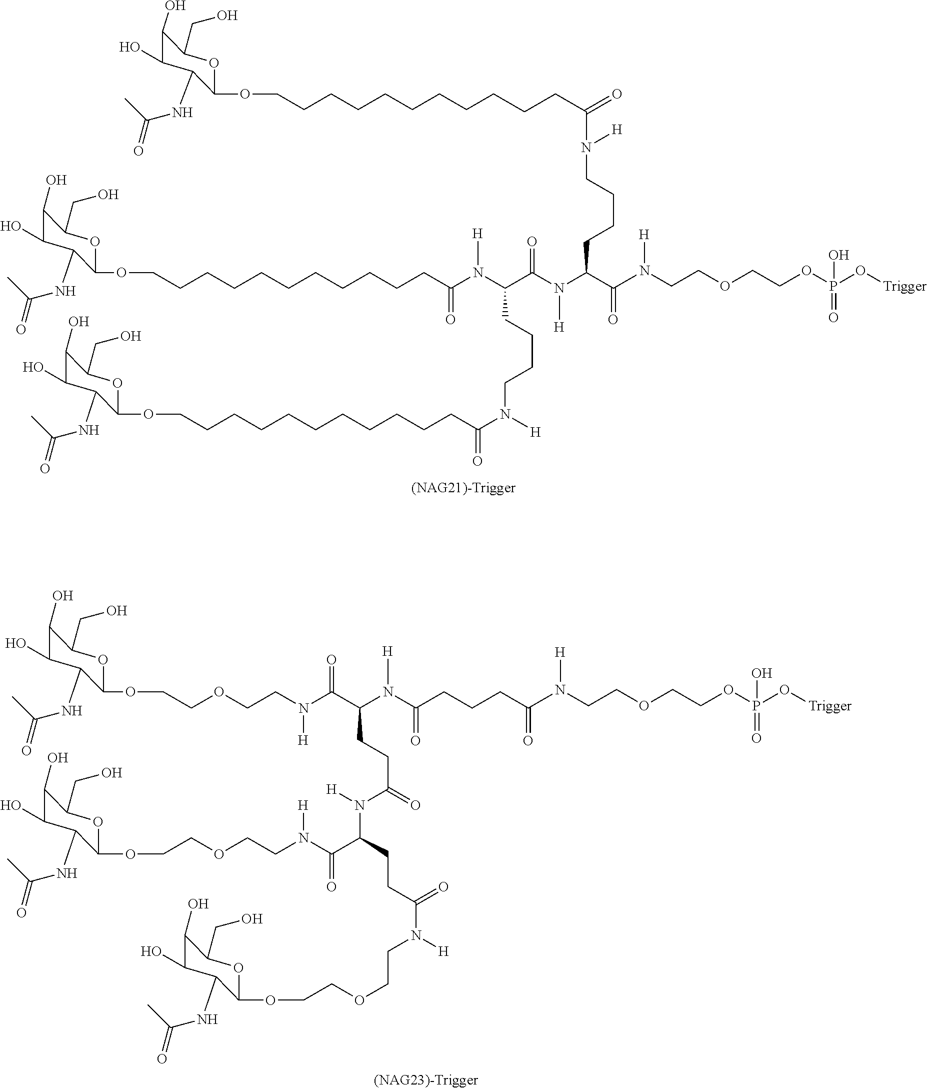

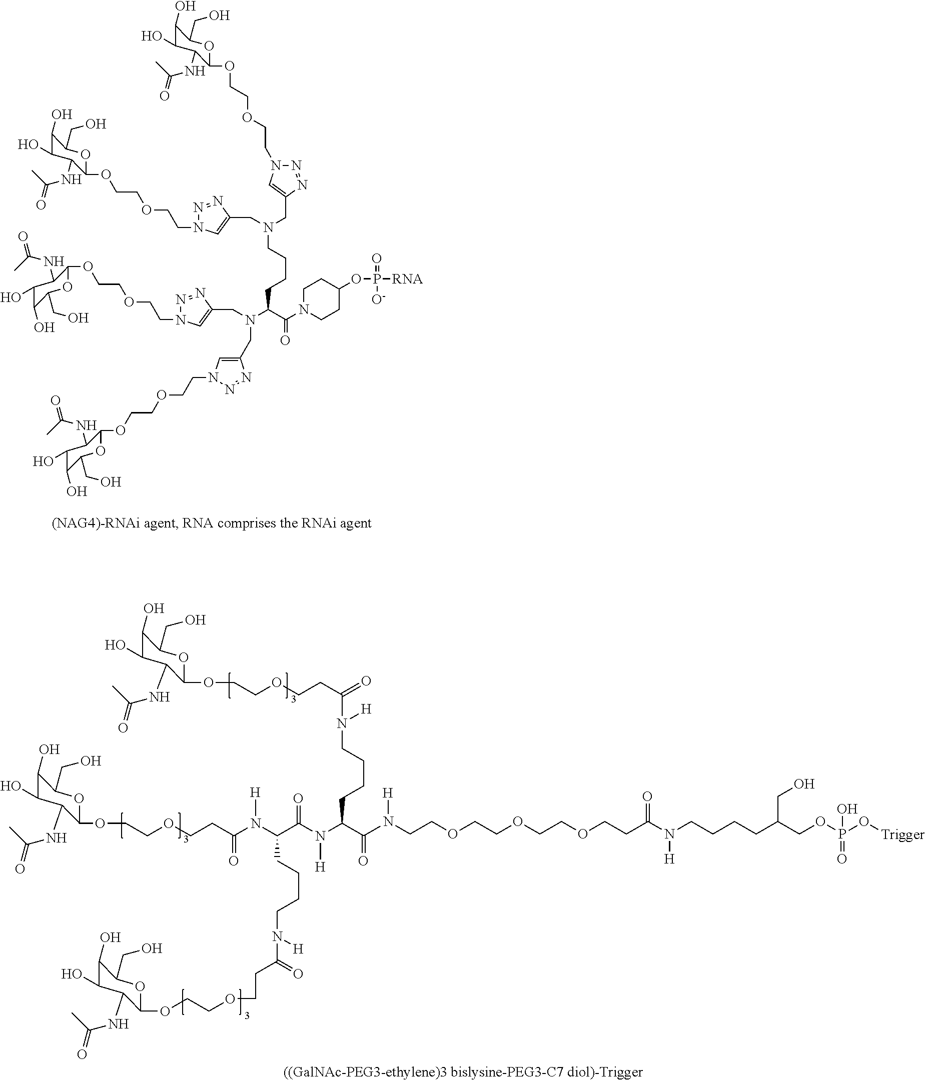

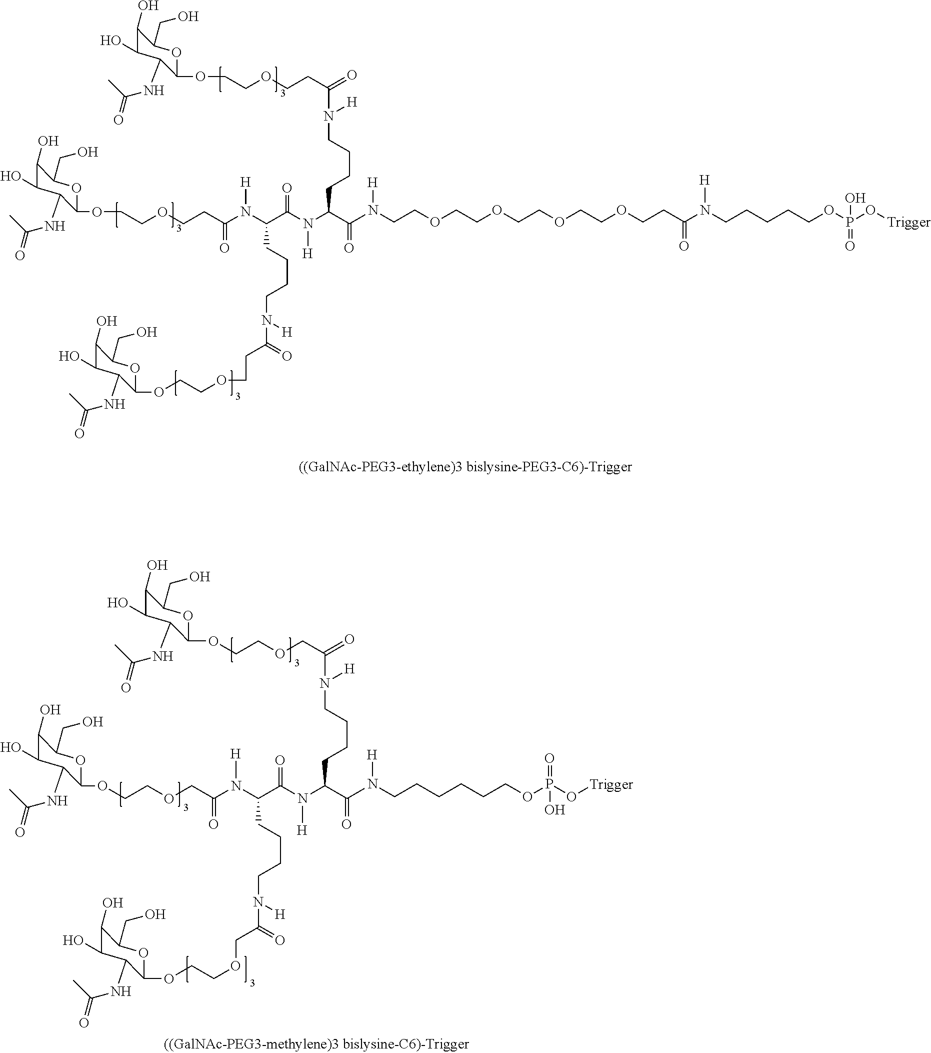

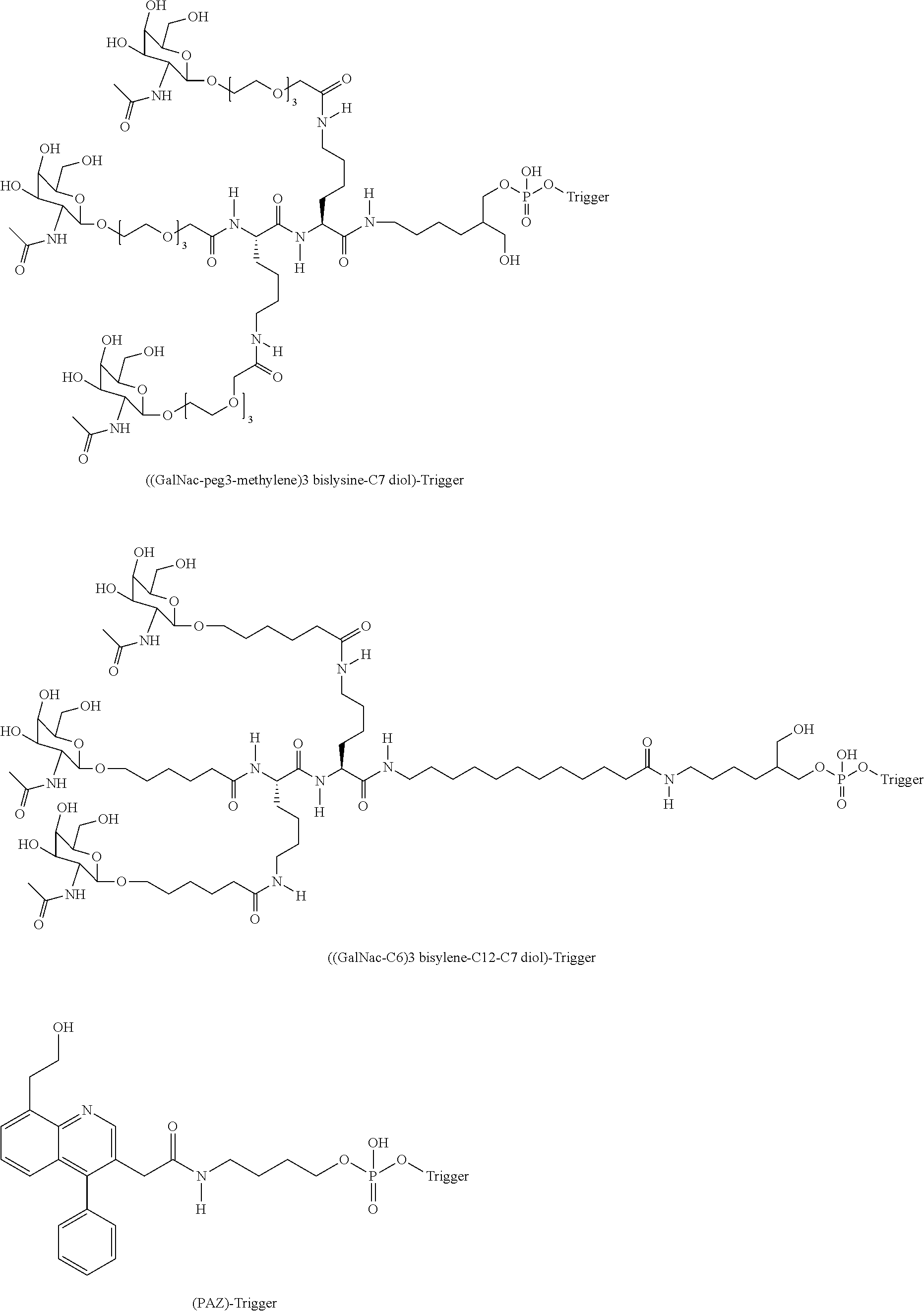

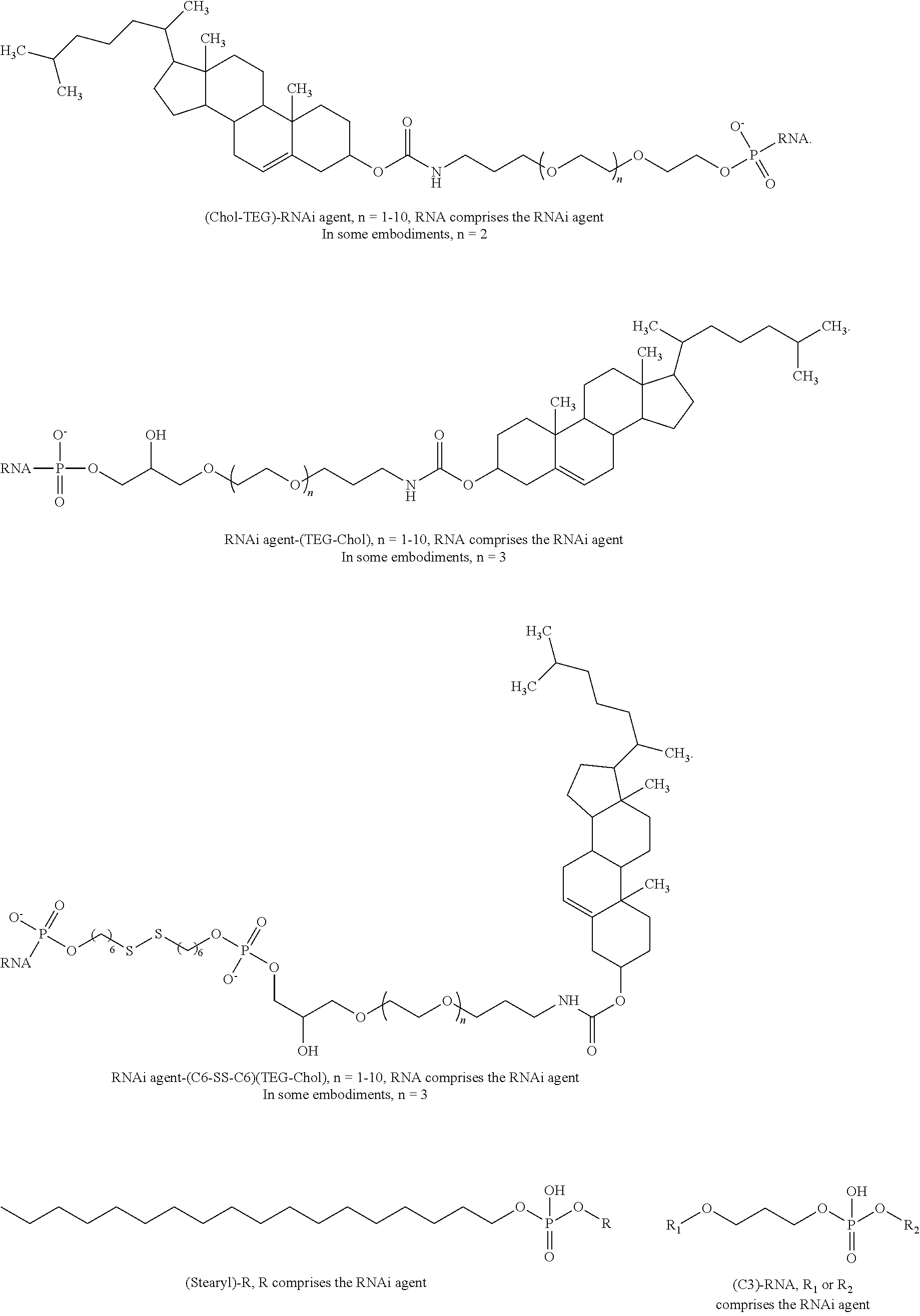

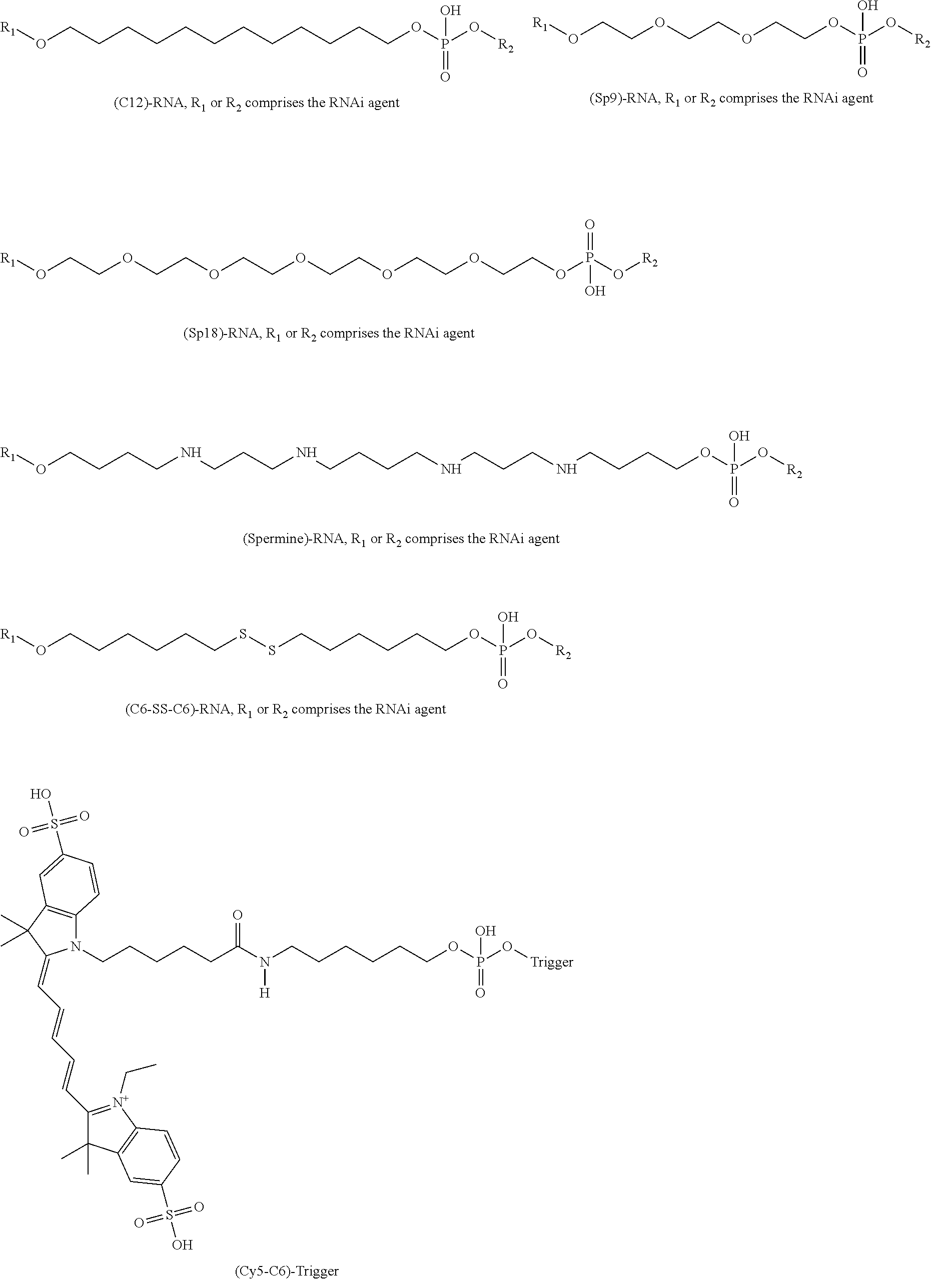

In some embodiments, a delivery vehicle may be used. A delivery vehicle is a compound which improves delivery of the RNAi agent to the cell. A delivery vehicle can be, but is not limited to: a polymer, such as an amphipathic polymer, membrane active polymer, a peptide, such as a melittin or melittin-like peptide, a reversibly modified polymer or peptide, or a lipid.

In some embodiments, the targeting group is a galactose cluster. In some embodiments, an RNAi agent as described herein is linked to a galactose cluster. As used herein, a galactose cluster comprises a molecule having two to four terminal galactose derivatives. As used herein, the term galactose derivative includes both galactose and derivatives of galactose having affinity for the asialoglycoprotein receptor equal to or greater than that of galactose. A terminal galactose derivative is attached to a molecule through its C-1 carbon. In some embodiments, a galactose cluster has three terminal galactosamines or galactosamine derivatives (such as N-acetyl-galactosamine) each having affinity for the asialoglycoprotein receptor. In some embodiments, a galactose cluster has three terminal N-acetyl-galactosamines. Other terms common in the art include tri-antennary galactose, tri-valent galactose and galactose trimer. It is known that tri-antennary galactose derivative clusters are bound to the ASGPr with greater affinity than bi-antennary or mono-antennary galactose derivative structures (Baenziger and Fiete, 1980, Cell, 22, 611-620; Connolly et al., 1982, J. Biol. Chem., 257, 939-945).

In some embodiments, a galactose cluster contains three galactose derivatives each linked to a central branch point. The galactose derivatives are attached to the central branch point through the C-1 carbons of the saccharides. In some embodiments, a galactose derivative is linked to the branch point via a linker or spacer. In some embodiments, the linker or spacer is a flexible hydrophilic spacer (U.S. Pat. No. 5,885,968; Biessen et al. J. Med. Chem. 1995 Vol. 39 p. 1538-1546), such as, but not limited to: a PEG spacer. In some embodiments, the PEG spacer is a PEG3 spacer. The branch point can be any small molecule which permits attachment of three galactose derivatives and further permits attachment of the branch point to the RNAi agent. Attachment of the branch point to the RNAi agent may occur through a linker or spacer. In some embodiments, the linker or spacer comprises a flexible hydrophilic spacer, such as, but not limited to: a PEG spacer. In some embodiments, a PEG spacer is a PEG3 spacer (three ethylene units). In other embodiments, the PEG spacer has 1 to 20 ethylene units (PEG₁ to PEG₂₀).

In some embodiments, a galactose derivative comprises an N-acetyl-galactosamine (GalNAc or NAG). Other saccharides having affinity for the asialoglycoprotein receptor may be selected from the list comprising: galactose, galactosamine, N-formyl-galactosamine, N-acetyl-galactosamine, N-propionyl-galactosamine, N-n-butanoylgalactosamine, and N-iso-butanoylgalactosamine. The affinities of numerous galactose derivatives for the asialoglycoprotein receptor have been studied (see for example: Iobst, S. T. and Drickamer, K. J.B.C. 1996, 271, 6686) or are readily determined using methods well known and commonly used in the art.

Nucleotides at positions 1-19 of the RNAi agents described herein are modified nucleotides. In some embodiments, nucleotides at positions 1-20 are modified nucleotides. In some embodiments, nucleotides at positions 1-21 are modified nucleotides. In some embodiments, nucleotides at positions 1-22 are modified nucleotides. In some embodiments, nucleotides at positions 1-23 are modified nucleotides. In some embodiments, nucleotides at positions 1-24 are modified nucleotides. In some embodiments, nucleotides at positions 1-25 are modified nucleotides. In some embodiments, nucleotides at positions 1-26 are modified nucleotides. In some embodiments, nucleotides at positions 1-24, and 26 are modified nucleotides. In some embodiments, nucleotides at positions 1-23, 25, and 26 are modified nucleotides. In some embodiments, nucleotides at positions 1-22, 24, and 26 are modified nucleotides.

Nucleotides at positions 1′-19′ of the RNAi agents described herein are modified nucleotides. In some embodiments, nucleotides at positions 1′-20′ are modified nucleotides. In some embodiments, nucleotides at positions 1′-21′ are modified nucleotides. In some embodiments, nucleotides at positions 1′-22′ are modified nucleotides. In some embodiments, nucleotides at positions 1′-23′ are modified nucleotides. In some embodiments, nucleotides at positions 1′-24′ are modified nucleotides. In some embodiments, nucleotides at positions 1′-24′ and 26′ are modified nucleotides. In some embodiments, nucleotides at positions 1′-22′, 24′ and 26′ are modified nucleotides. In some embodiments, nucleotides at positions 1′-22′, 25′, and 26′ are modified nucleotides. In some embodiments, nucleotides at positions 1′-23′ and 26′ are modified nucleotides.

In some embodiments, nucleotides at positions 1′-22′, 26′, 1-22, and 26 are modified nucleotides. In some embodiments, position 1 is an inverted deoxynucleotide, a 2′-fluoro nucleotide (2′-F), a 2′-O-methyl nucleotide (2′-OMe), or a 2′-methoxyethoxy nucleotide (2′-MOE). In some embodiments, position 1′ is a 2′-F nucleotide, an inverted deoxynucleotide, a 2′-OMe nucleotide, or a 2′-MOE nucleotide.

The RNAi agents described herein contain at least one ribonucleotide. Ribonucleotides include ribopurines (A, G) and ribopyrimidines (C, U).

The RNAi agents described herein are contain modified nucleotides. A nucleotide base (or nucleobase) is a heterocyclic pyrimidine or purine compound which is a constituent of all nucleic acids and includes adenine (A), guanine (G), cytosine (C), thymine (I), and uracil (U). As used herein, “G,” “g”, “C,” “c”, “A”, “a”, “U”, “u”, and “T”, each generally stand for a nucleobase, nucleoside, nucleotide or nucleotide mimic that contains guanine, cytosine, adenine, uracil and thymidine as a base, respectively. Also as used herein, the term “nucleotide” may include a modified nucleotide or nucleotide mimic, abasic site, or a surrogate replacement moiety. As used herein, a “modified nucleotide” is a nucleotide, nucleotide mimic, abasic site, or a surrogate replacement moiety other than a ribonucleotide (2′-hydroxyl nucleotide). In one embodiment a modified nucleotide comprises a 2′-modified nucleotide (i.e. a nucleotide with a group other than a hydroxyl group at the 2′ position of the five-membered sugar ring). Ribonucleotide are represented herein as “N” (capital letter without further notation). Modified nucleotides include, but are not limited to: 2′-modified nucleotides, 2′-O-methyl nucleotides (represented herein as a lower case letter ‘n’ in a nucleotide sequence), 2′-deoxy-2′-fluoro nucleotides (represented herein as Nf, also represented herein as 2′-fluoro nucleotide), 2′-deoxy nucleotides (represented herein as dN), 2′-methoxyethyl (2′-O-2-methoxylethyl) nucleotides (represented herein as NM or 2′-MOE), 2′-amino nucleotides, 2′-alkyl nucleotides, 3′ to 3′ linkages (inverted) nucleotides (represented herein as invdN, invN, invn, invX), non-natural base comprising nucleotides, bridged nucleotides, peptide nucleic acids, 2′,3′-seco nucleotide mimics (unlocked nucleobase analogues, represented herein as N_{UNA} or NUNA), locked nucleotides (represented herein as N_{LNA} or NLNA), 3′-O-Methoxy (2′ internucleotide linked) nucleotide (represented herein as 3′-OMen), 2′-F-Arabino nucleotides (represented herein as NfANA or Nf_{ANA}), morpholino nucleotides, vinyl phosphonate deoxyribonucleotide (represented herein as vpdN), vinyl phosphonate nucleotides, and abasic nucleotides (represented herein as X or Ab). It is not necessary for all positions in a given compound to be uniformly modified. Conversely, more than one modification may be incorporated in a single RNAi agent or even in a single nucleotide thereof. The RNAi agent sense strands and antisense strands described herein may be synthesized and/or modified by methods known in the art. Modification at each nucleotide is independent of modification of the other nucleotides.

Modified nucleobases include synthetic and natural nucleobases, such as 5-substituted pyrimidines, 6-azapyrimidines and N-2, N-6 and O-6 substituted purines, including 2-aminopropyladenine, 5-propynyluracil and 5-propynylcytosine, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

Nucleotides of an RNAi agent described herein may be linked by phosphate-containing or non-phosphate-containing covalent internucleoside linkages. Modified internucleoside linkages or backbones include, for example, phosphorothioates, 5′-phosphorothioate group (represented herein as a lower case ‘s’ before a nucleotide, as in sN, sn, sNf, or sdN), chiral phosphorothioates, thiophosphate, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkyl-phosphonates, thionoalkylphosphotriesters, and boranophosphates having normal linkages, linked analogs of these, and those having inverted polarity wherein the adjacent pairs of nucleoside units are linked to 5′-3′ or to 5′-2′. Various salts, mixed salts and free-acid forms are also included.

Modified internucleoside linkages or backbones that do not include a phosphorus atom therein (i.e., oligonucleosides) have backbones that are formed by short chain alkyl or cycloalkyl inter-sugar linkages, mixed heteroatom and alkyl or cycloalkyl inter-sugar linkages, or one or more short chain heteroatomic or heterocyclic inter-sugar linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

The herein described RNAi agents have blunt ends. As used herein, the terminal nucleotides of a blunt end may be complementary or may not be complementary. As used herein a frayed end refers to an end of a blunt end in which the terminal nucleotides of the two annealed strands are not complementary (i.e. do not form a non-complementary base-pair).

RNA interference (RNAi) agents (also dsRNAi triggers, RNAi triggers, or triggers) are double strand oligonucleotides capable of inducing RNA interference through interaction with the RNA interference pathway machinery (RNA-induced silencing complex or RISC) of mammalian cells. RNA interference leads to degradation or inhibits translation of messenger RNA (mRNA) transcripts of a target mRNA in a sequence specific manner.

An siRNA agent comprises a sense strand and an antisense strand that are at least partially complementary (at least 70% complementary) to each other. The antisense strand contains a region having a sequence that is perfectly complementary (100% complementary) or at least substantially complementary (at least 85% complementary) to a sequence in a target mRNA. This region of perfect or substantial complementarity is typically 15-25 nucleotides in length and occurs at or near the 5′ end of the antisense strand.

The sense and antisense strands of the described RNAi agents are synthesized using methods commonly used in the art. Double strand RNAi agents can be formed by annealing an antisense strand with a sense strand.

The described RNAi agents and methods can be used to treat a subject having a disease or disorder that would benefit from reduction or inhibition expression of the target mRNA. The subject is administered a therapeutically effective amount of any one or more of the RNAi agents. The subject can be a human, patient, or human patient. The described RNAi agents can be used to provide a method for the therapeutic treatment of diseases. Such methods comprise administration of a described herein RNAi agent to a human being or animal.

We describe compositions and methods for inhibiting expression of a target mRNA in a cell, group of cells, tissue, or subject, comprising: administering to the subject a therapeutically effective amount of a herein described RNAi agent thereby inhibiting the expression of a target mRNA in the subject. Silence, reduce, inhibit, down-regulate, or knockdown gene expression, in as far as they refer to a target RNA, means that the expression of mRNA, as measured by the level of mRNA in a cell, group of cells, tissue, or subject, or the level of polypeptide, protein or protein subunit translated from the mRNA in a cell, group of cells, or tissue, or subject in which the target mRNA gene is transcribed, is reduced when the cell, group of cells, or tissue, or subject is treated with the described RNAi agents as compared to a second cell, group of cells, or tissue, or subject substantially which has not or have not been so treated.

In some embodiments, we describe pharmaceutical compositions comprising at least one of the described RNAi agents. These pharmaceutical compositions are particularly useful in the inhibition of the expression of a target mRNA in a cell, a group of cells, a tissue, or an organism. The described pharmaceutical compositions can be used to treat a subject having a disease or disorder that would benefit from reduction or inhibition in expression of the target mRNA. The described pharmaceutical compositions can be used to treat a subject at risk of developing a disease or disorder that would benefit from reduction or inhibition in expression of the target mRNA. In one embodiment, the method comprises administering a composition comprising an RNAi agent described herein to a subject to be treated. In some embodiments a pharmaceutical composition comprises one or more pharmaceutically acceptable excipients (including vehicles, carriers, diluents, and/or delivery polymers).

In some embodiments, the described RNAi agents are used for treating, preventing, or managing clinical presentations associated with expression of a target mRNA. In some embodiments, a therapeutically or prophylactically effective amount of one or more RNAi agents is administered to a subject in need of such treatment, prevention or management.

The described RNAi agents and methods can be used to treat or prevent at least one symptom in a subject having a disease or disorder that would benefit from reduction or inhibition in expression of a target mRNA. In some embodiments, the subject is administered a therapeutically effective amount of one or more RNAi agents thereby treating the symptom. In other embodiments, the subject is administered a prophylactically effective amount of one or more of RNAi agents thereby preventing the at least one symptom.

In some embodiments, expression of a target mRNA in a subject to whom an RNAi agent is administered is reduced by at least about 5%, 10%, 15%, 20% 25%, 30%, 35%, 40%, 45%, 50%, 55%, 60%, 65%, 70%, 75%, 80%, 85%, 90%, 95%, or 98% relative to the subject not receiving the RNAi agent. The gene expression level in the subject may be reduced in a cell, group of cells, and/or tissue of the subject. In some embodiments, the level of mRNA is reduced. In other embodiments, the expressed protein level is reduced. Reduction in expression, mRNA levels, or protein levels can be assessed by any methods known in the art. Reduction or decrease in mRNA level and/or protein level are collectively referred to herein as a reduction or decrease in target RNA or inhibiting or reducing the expression of target mRNA.

“Introducing into a cell”, when referring to an RNAi agent, means functionally delivering the RNAi agent into a cell. By functional delivery, it is meant that the RNAi agent is delivered to the cell and has the expected biological activity, sequence-specific inhibition of gene expression.

The route of administration is the path by which an RNAi agent is brought into contact with the body. In general, methods of administering drugs and nucleic acids for treatment of a mammal are well known in the art and can be applied to administration of the compositions described herein. The herein described RNAi agents can be administered via any suitable route in a preparation appropriately tailored to the particular route. Thus, herein described RNAi agents can be administered by injection, for example, intravenously, intramuscularly, intracutaneously, subcutaneously, or intraperitoneally. Accordingly, in some embodiments, pharmaceutical compositions may comprise one or more pharmaceutically acceptable excipients.

In one embodiment, RNAi agents described herein can be formulated for administration to a subject.

The RNAi agents or compositions described herein can be delivered to a cell, group of cells, tumor, tissue, or subject using oligonucleotide delivery technologies known in the art. In general, any suitable method recognized in the art for delivering a nucleic acid molecule (in vitro or in vivo) can be adapted for use with a herein described RNAi agents. For example, delivery can be by local administration, (e.g., direct injection, implantation, or topical administering), systemic administration, or subcutaneous, intravenous, oral, intraperitoneal, or parenteral routes, including intracranial (e.g., intraventricular, intraparenchymal and intrathecal), intramuscular, transdermal, airway (aerosol), nasal, rectal, or topical (including buccal and sublingual) administration, In certain embodiments, the compositions are administered by subcutaneous or intravenous infusion or injection.

The RNAi agents can be combined with lipids, nanoparticles, polymers, liposomes, micelles. DPCs or other delivery systems available in the art. The RNAi agents can also be chemically conjugated to targeting moieties, lipids (including, but not limited to cholesterol and cholesteryl derivative), nanoparticles, polymers, liposomes, micelles, DPCs (WO 2015/021092, WO 2000/053722, WO 2008/0022309, WO 2013/158141, and WO 2011/104169), or other delivery systems available in the art.

As used herein, a “pharmaceutical composition” comprises a pharmacologically effective amount of at least one kind of RNAi agent and one or more a pharmaceutically acceptable excipients. Pharmaceutically acceptable excipients (excipients) are substances other than the Active Pharmaceutical ingredient (API, therapeutic product, e.g., RNAi agent) that have been appropriately evaluated for safety and are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use.

Excipients include, but are not limited to: absorption enhancers, anti-adherents, anti-foaming agents, anti-oxidants, binders, binders, buffering agents, carriers, coating agents, colors, delivery enhancers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents. A pharmaceutically acceptable excipient may or may not be an inert substance.

The pharmaceutical compositions can contain other additional components commonly found in pharmaceutical compositions. The pharmaceutically-active materials may include, but are not limited to: anti-pruritics, astringents, local anesthetics, or anti-inflammatory agents (e.g., antihistamine, diphenhydramine, etc.). It is also envisaged that cells, tissues or isolated organs that express or comprise the herein defined RNAi agents may be used as “pharmaceutical compositions”. As used herein, “pharmacologically effective amount,” “therapeutically effective amount,” or simply “effective amount” refers to that amount of an RNAi agent to produce the intended pharmacological, therapeutic or preventive result.

In some embodiments, an RNAi agent is conjugated to a delivery polymer. In some embodiments, the delivery polymer is a reversibly masked/modified amphipathic membrane active polyamine.

The described RNAi agents can be used to provide therapeutic treatments of diseases. Such uses comprise administration of RNAi agent to a human being or animal. For treatment of disease or for formation of a medicament or composition for treatment of a disease, a herein described RNAi agent can be combined with an excipient or with a second therapeutic or treatment including, but not limited to: a second RNAi agent or other RNAi agent, a small molecule drug, an antibody, an antibody fragment, and a vaccine.

The described RNAi agents and pharmaceutical compositions comprising RNAi agents disclosed herein may be packaged separately or included in a kit, container, pack, or dispenser. The RNAi agents may be packaged in pre-filled syringes or vials.

The above provided embodiments are now illustrated with the following, non-limiting examples.

### EXAMPLES

### Example 1

### RNAi Agent Synthesis

### A) Synthesis.

RNAi agents were synthesized according to phosphoramidite technology on solid phase used in oligonucleotide synthesis. Depending on the scale either a MerMade96E (Bioautomation) or a MerMade12 (Bioautomation) was used. Syntheses were performed on a solid support made of controlled pore glass (CPG, 500 Å or 600 Å, obtained from Prime Synthesis, Aston, Pa., USA). All DNA, 2′-modified RNA, and UNA phosphoramidites were purchased from Thermo Fisher Scientific (Milwaukee, Wis., USA). Specifically, the following 2′-O-Methyl phosphoramidites were used: (5′-O-dimethoxytrityl-N⁶-(benzoyl)-2′-O-methyl-adenosine-3′-O-(2-cyanoethyl-N,N-diisopropy-lamino) phosphoramidite, 5′-O-dimethoxy-trityl-N⁴-(acetyl)-2′-O-methyl-cytidine-3′-O-(2-cyanoethyl-N,N-diisopropylamino) phosphoramidite, (5′-O-dimethoxytrityl-N²-(isobutyryl)-2′-O-methyl-guanosine-3′-O-(2-cyano-ethyl-N,N-diisopropylamino)phosphoramidite, and 5′-O-dimethoxy-trityl-2′-O-methyl-undine-3′-O-(2-cyanoethyl-N,N-diisopropylamino)phosphoramidite. The 2′-Deoxy-2′-fluoro-phosphor-amidites carried the same protecting groups as the 2′-O-methyl RNA amidites. The following UNA phosphoramidites were used: 5′-(4,4′-Dimethoxytrityl)-N-benzoyl-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diisopropyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxytrityl)-2′,3′-seco-uridine, 2′-benzoyl-3′-[(2-cyanoethyl)-(N,N-diisopropyl)]-phosphoramidite. All amidites were dissolved in anhydrous acetonitrile (50 mM) and molecular sieves (3 Å) were added. In order to introduce the TEG-Cholesterol at the 5′-end of the oligomers, the 1-Dimethoxytrityloxy-3-O—(N-cholesteryl-3-aminopropyl)-triethyleneglycol-glyceryl-2-O-(2-cyanoethyl)-(N,N,-diisopropyl)-phosphoramidite from Glen Research (Sterling, Va., USA) was employed. The 5′-modifications were introduced without any modification of the synthesis cycle. 5-Benzylthio-1H-tetrazole (BTT, 250 mM in acetonitrile) was used as activator solution. Coupling times were 10 min (RNA), 180 sec (Cholesterol), 90 sec (2′OMe and UNA), and 60 sec (2′F and DNA). In order to introduce phosphorothioate linkages, a 100 mM solution of 3-phenyl 1,2,4-dithiazoline-5-one (POS, obtained from PolyOrg, Inc., Leominster, Mass., USA) in anhydrous Acetonitrile was employed.

### B. Cleavage and Deprotection of Support Bound Oligomer.

After finalization of the solid phase synthesis, the dried solid support was treated with a 1:1 volume solution of 40 wt. % methylamine in water and 28% ammonium hydroxide solution (Aldrich) for two hours at 30° C. The solution was evaporated and the solid residue was reconstituted in water (see below).

### C. Purification.

Crude Cholesterol containing oligomers were purified by reverse phase HPLC using a Waters XBridge BEH300 C4 5u Prep column and a Shimadzu LC-8 system. Buffer A was 100 mM TEAA, pH 7.5 and contained 5% Acetonitrile and buffer B was 100 mM TEAA and contained 95% Acetonitrile. UV traces at 260 nm were recorded. Appropriate fractions were then run on size exclusion HPLC using a GE Healthcare XK 16/40 column packed with Sephadex G-25 medium with a running buffer of 100 mM ammonium bicarbonate, pH 6.7 and 20% Acetonitrile. Other crude oligomers were purified by anionic exchange HPLC using a TKSgel SuperQ-5PW 13u column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC as described for Cholesterol containing oligomers.

### D. Annealing.

Complementary strands were mixed by combining equimolar RNA solutions (sense and antisense) in 0.2×PBS (Phosphate-Buffered Saline, 1×, Corning, Cellgro) to form the RNAi agents. This solution was placed into a thermomixer at 70° C., heated to 95° C., held at 95° C. for 5 min, and cooled to room temperature slowly. Some RNAi agents were lyophilized and stored at −15 to −25° C. Duplex concentration was determined by measuring the solution absorbance on a UV-Vis spectrometer in 0.2×PBS. The solution absorbance at 260 nm was then multiplied by a conversion factor and the dilution factor to determine the duplex concentration. Unless otherwise stated, all conversion factor was 0.037 mg/(mL·cm). For some experiments, a conversion factor was calculated from an experimentally determined extinction coefficient.

TABLE 1
Exemplary blunt ended 26 mer RNAi agent sequences

| Duplex ID | SEQ ID | Antisense Sequence | SEQ ID | Sense Sequence |
|---|---|---|---|---|
| No. | No. | (5′→3′) | No. | (5′→3′) |

Exemplary Factor 12 26 mer RNAi agent sequences

| | | | | |
|---|---|---|---|---|
| AD01457 | 1 | TGAGAAGCUGAGGCUCAAAGCACUAU | 31 | UAUAUGCUUUGAGCCUCAGCUUCUCA |
| AD01459 | 2 | TGAGAAGCUGAGGCUCAAAGCAUAUA | 32 | UAUAUGCUUUGAGCCUCAGCUUCUCA |
| AD01520 | 3 | TGGUCUUUCACUUUCUUGGGCUCUAU | 33 | UAUAUGCCCAAGAAAGUGAAAGACCA |
| AD01537 | 4 | TCACUUUCUUGGGCUCCAAACAGUAU | 34 | UAUAUGUUUGGAGCCCAAGAAAGUGA |
| AD01538 | 5 | TUCACUUUCUUGGGCUCCAAACAUAU | 35 | UAUAUUUUGGAGCCCAAGAAAGUGAA |
| AD01539 | 6 | TUUCACUUUCUUGGGCUCCAAACUAU | 36 | UAUAUUUGGAGCCCAAGAAAGUGAAA |
| AD01540 | 7 | TUUUCACUUUCUUGGGCUCCAAAUAU | 37 | UAUAUUGGAGCCCAAGAAAGUGAAAA |
| AD01541 | 8 | TAGCUGAGGCUCAAAGCACUUCUUAU | 38 | UAUAUAAGUGCUUUGAGCCUCAGCUA |
| AD01542 | 9 | TGAAGCUGAGGCUCAAAGCACUUUAU | 39 | UAUAUGUGCUUUGAGCCUCAGCUUCA |
| AD01543 | 10 | TUUGUUGCGGUCACCACAGCCCGUAU | 40 | UAUAUGGCUGUGGUGACCGCAACAAA |
| AD01544 | 11 | TGCUUGUUGCGGUCACCACAGCCUAU | 41 | UAUAUCUGUGGUGACCGCAACAAGCA |
| AD01545 | 12 | TGGCUUGUUGCGGUCACCACAGCUAU | 42 | UAUAUUGUGGUGACCGCAACAAGCCA |
| AD01577 | 13 | TGGUCUUUCACUUUCUUGGGCUCUAU | 43 | UAUUAGCCCAAGAAAGUGAAAGACCA |
| AD01579 | 14 | TGGUCUUUCACUUUCUUGGGCUCUAU | 44 | UAUAAGCCCAAGAAAGUGAAAGACCA |
| AD02068 | 15 | UGGUCUUUCACUUUCUUGGGCUCUAU | 45 | UAUAUGCCCAAGAAAGUGAAAGACCA |
| AD02765 | 16 | UGGUCUUUCACUUUCUUGGGCUCUAU | 46 | UUAGAGCCCAAGAAAGUGAAAGACCA |
| AD02766 | 17 | UGGUCUUUCACUUUCUUGGGCUCUAU | 47 | UUAUUGCCCAAGAAAGUGAAAGACCA |
| AD02767 | 18 | UGGUCUUUCACUUUCUUGGGCUCUAU | 48 | UUGAUGCCCAAGAAAGUGAAAGACCA |
| AD02769 | 19 | UGGUCUUUCACUUUCUUGGGCUCUAU | 49 | UAUGAGCCCAAGAAAGUGAAAGACCA |
| AD02772 | 20 | TGGUCUUUCACUUUCUUGGGCUCUAU | 50 | AUAGAGCCCAAGAAAGUGAAAGACCA |
| AD01610 | 21 | UGAAGCUGAGGCUCAAAGCACUUUAU | 51 | UAUAUGUGCUUUGAGCCUCAGCUUCA |
| AD01775 | 22 | TGAAGCUGAGGCUCAAAGCACUUUAU | 52 | UAUAUGUGCUUUGAGCCUCAGCUUCA |
| AD01856 | 23 | UGGUCUUUCACUUUCUUGGGCUCUAU | 53 | UAUAUGCCCAAGAAAGUGAAAGACCA |
| AD01975 | 24 | UGGUCUUUCACUUUCUUGGGCTCUAU | 54 | UAUAUGCCCAAGAAAGUGAAAGACCA |
| AD01994 | 25 | UGGUCUUUCACUUUCUUGGGCUCUAU | 55 | UAUAUGCCCAAGAAAGUGAAAGAUAU |
| AD02665 | 26 | UAUGGUCUUUCACUUUCUUGGGCUCU | 56 | UAUGCCCAAGAAAGUGAAAGACCUAU |
| AD02666 | 27 | UAUGGUCUUUCACUUUCUUGGGCUCU | 57 | UAUGCCCAAGAAAGUGAAAGACCAAU |
| AD02703 | 28 | UAUGGUCUUUCACUUUCUUGGGCUCU | 58 | UAUGCCCAAGAAAGUGAAAGACCAUU |
| AD02704 | 29 | UAUGGUCUUUCACUUUCUUGGGCUCU | 59 | UAUGCCCAAGAAAGUGAAAGACCAUA |
| AD02809 | 30 | UAUGGUCUUUCACUUUCUUGGGCUCU | 60 | UAUGCCCAAGAAAGUGAAAGACCUAU |

Exemplary LPA 26 mer RNAi agent sequences

| | | | | |
|---|---|---|---|---|
| AD01466 | 61 | TGACACCUGAUUCUGUUUCUGAGUAU | 79 | UAUAUCAGAAACAGAAUCAGGUGUCA |
| AD01462 | 62 | TGAGAAUGAGCCUCGAUAACUCUUAU | 80 | UAUAUAGUUAUCGAGGCUCAUUCUCA |
| AD02664 | 63 | TGAGAAUGAGCCUCGAUAACUCUUAU | 81 | UAUAUAGUUAUCGAGGCUCAUUCUCA |
| AD01530 | 64 | TGCGUCUGAGCAUUGUGUCAGGUUAU | 82 | UAUAUCUGACACAAUGCUCAGACGCA |
| AD01531 | 65 | TUGCGUCUGAGCAUUGUGUCAGGUAU | 83 | UAUAUUGACACAAUGCUCAGACGCAA |
| AD01534 | 66 | TAAGGGCGAAUCUCAGCAUCUGGUAU | 84 | UAUAUAGAUGCUGAGAUUCGCCCUUA |
| AD01532 | 67 | TGAGAAUGAGCCUCGAUAACUCUUAU | 85 | UAUAUAGUUAUCGAGGCUCAUUCUCA |
| AD01981 | 68 | UGAGAAUGAGCCUCGAUAACUCTUAU | 86 | UAUAUAGUUAUCGAGGCUCAUUCUCA |
| AD01979 | 69 | UGAGAAUGAGCCUCGAUAACUCUUAU | 87 | UAUAUAGUUAUCGAGGCUCAUUCUCA |
| AD02435 | 70 | UGAGAAUGAGCCUCGAUAACUCUUAU | 88 | UAUAUAGUUAUCGAGGCUCAUUCUCA |
| AD02619 | 71 | UGAGAAUGAGCCUCGAUAACUCUUAU | 89 | UAUAUAGUUAUCGAGGCUCAUUCUCA |
| AD01533 | 72 | TGACACCUGAUUCUGUUUCUGAGUAU | 90 | UAUAUCAGAAACAGAAUCAGGUGUCA |
| AD01772 | 73 | UGACACCUGAUUCUGUUUCUGAGUAU | 91 | UAUAUCAGAAACAGAAUCAGGUGUCA |
| AD01773 | 74 | UGACACCUGAUUCUGUUUCUGAGUAU | 92 | UAUAUCAGAAACAGAAUCAGGUGUCA |
| AD01774 | 75 | UGACACCUGAUUCUGUUUCUGAGUAU | 93 | UAUAUCAGAAACAGAAUCAGGUGUCA |
| AD02714 | 76 | TCGUAUAACAAUAAGGGGCUGCCUAU | 94 | UAUAUCAGCCCCUUAUUGUUAUACGA |
| AD02552 | 77 | UCGUAUAACAAUAAGGGGCUGCCUAU | 95 | UAUAUCAGCCCCUUAUUGUUAUACGA |
| AD02752 | 78 | UCGUAUAACAAUAAGGGGCUGCCUAU | 96 | UAUAUCAGCCCCUUAUUGUUAUACGA |

Exemplary Hif2alpha 26 mer RNAi agent sequences

| | | | | |
|---|---|---|---|---|
| AD01295 | 97 | TUUCAUGAAAUCGUUACGUUGGCUAU | 102 | UAUAUCAACGUAACGAUUUCAUGAAA |
| AD01293 | 98 | TUUCAUGAAAUCGUUACGUUGGCUGU | 103 | UAUAUCAACGUAACGAUUUCAUGAAA |
| AD01296 | 99 | TUUCAUGAAAUCGUUACGUUGGCUTT | 104 | UAUAUCAACGUAACGAUUUCAUGAAA |
| AD01411 | 100 | TUUCAUGAAAUCGUUACGUCGGCUAU | 105 | UAUAUCGACGUAACGAUUUCAUGAAA |
| AD01294 | 101 | TUUCAUGAAAUCGUUACGUCGGCUGU | 106 | UAUAUCGACGUAACGAUUUCAUGAAA |

Exemplary FVII RNAi agent sequences

| | | | | |
|---|---|---|---|---|
| AD01149 | 107 | TGAGUUGGCACGCCUUUGCTT | 109 | UGUGCAAAGGCGUGCCAACUCAT |
| AD01259 | 108 | TGAGUUGGCACGCCUUUGCTT | 110 | UGUGCAAAGGCGUGCCAACUCAT |

TABLE 2
Exemplary modified blunt-ended 26 mer RNAi agent sequences.

| | | | |
|---|---|---|---|
| | | SEQ | |
| Duplex ID | strand ID | ID | |
| No. | no. | No. | modified antisense strand |

Exemplary Factor 12 RNAi agents and sequences

| | | | |
|---|---|---|---|
| | AM02395-AS | 111 | dTsGfsaGfaAfgCfuGfaGfgCfuCfaAfaGfcascsuAu |
| AD01457 | AM02436-SS | 112 | (Chol-TEG)uAuAusGfscUfuUfgAfgCfcUfcAfgCfuUfcUfc(invdA) |
| | AM02438-AS | 113 | dTsGfsaGfaAfgCfuGfaGfgCfuCfaAfaGfcauAfsusa |
| AD01459 | AM02436-SS | 114 | (Chol-TEG)uAuAusGfscUfuUfgAfgCfcUfcAfgCfuUfcUfc(invdA) |
| | AM02507-AS | 115 | dTsGfgUfcUfuUfcAfcUfuUfCfuuGfgGfcucuAu |
| AD01520 | AM02500-SS | 116 | (Chol-TEG)uAuAusGfcCfcAfAfgaAfaGfuGfaAfaGfaCfc(invdA) |
| | AM02464-AS | 117 | dTsCfsaCfuUfuCfuUfgGfgCfuCfcAfaAfcAfsgsuAu |
| AD01537 | AM02513-SS | 118 | uAuAusGfsuUfuGfgAfgCfcCfaAfgAfaAfgUfgAf(C6-SS-Alk-Me) |
| | AM02466-AS | 119 | dTsUfscAfcUfuUfcUfuGfgGfcUfcCfaAfaCfsasuAu |
| AD01538 | AM02514-SS | 120 | uAuAusUfsuUfgGfaGfcCfcAfaGfaAfaGfuGfaAf(C6-SS-Alk-Me) |
| | AM02468-AS | 121 | dTsUfsuCfaCfuUfuCfuUfgGfgCfuCfcAfaAfscsuAu |
| AD01539 | AM02515-SS | 122 | uAuAusUfsuGfgAfgCfcCfaAfgAfaAfgUfgAfaAf(C6-SS-Alk-Me) |
| | AM02470-AS | 123 | dTsUfsuUfcAfcUfuUfcUfuGfgGfcUfcCfaAfsasuAu |
| AD01540 | AM02516-SS | 124 | uAuAusUfsgGfaGfcCfcAfaGfaAfaGfuGfaAfaAf(C6-SS-Alk-Me) |
| | AM02472-AS | 125 | dTsAfsgCfuGfaGfgCfuCfaAfaGfcAfcUfuCfsusuAu |
| AD01541 | AM02517-SS | 126 | uAuAusAfsaGfuGfcUfuUfgAfgCfcUfcAfgCfuAf(C6-SS-Alk-Me) |
| | AM02474-AS | 127 | dTsGfsaAfgCfuGfaGfgCfuCfaAfaGfcAfcUfsusuAu |
| AD01542 | AM02518-SS | 128 | uAuAusGfsuGfcUfuUfgAfgCfcUfcAfgCfuUfcAf(C6-SS-Alk-Me) |
| | AM02476-AS | 129 | dTsUfsuGfuUfgCfgGfuCfaCfcAfcAfgCfcCfsgsuAu |
| AD01543 | AM02519-SS | 130 | uAuAusGfsgCfuGfuGfgUfgAfcCfgCfaAfcAfaAf(C6-SS-Alk-Me) |
| | AM02478-AS | 131 | dTsGfscUfuGfuUfgCfgGfuCfaCfcAfcAfgCfscsuAu |
| AD01544 | AM02520-SS | 132 | uAuAusCfsuGfuGfgUfgAfcCfgCfaAfcAfaGfcAf(C6-SS-Alk-Me) |
| | AM02480-AS | 133 | dTsGfsgCfuUfgUfuGfcGfgUfcAfcCfaCfaGfscsuAu |
| AD01545 | AM02521-SS | 134 | uAuAusUfsgUfgGfuGfaCfcGfcAfaCfaAfgCfcAf(C6-SS-Alk-Me) |
| | AM02631-AS | 135 | dTsGfgUfcUfuUfcAfcuUfUfcUfuGfgGfcusCUAU |
| AD01577 | AM02634-SS | 136 | (Chol-TEG)UAUUAGfscCfcAfaGfaaAfGfuGfaAfaGfaCfc(invdA) |
| | AM02632-AS | 137 | dTsGfgUfcUfuUfcAfcuUfUfcUfuGfgGfcusCuAfu |
| AD01579 | AM02635-SS | 138 | (Chol-TEG)UfaUfaAGfscCfcAfaGfaaAfGfuGfaAfaGfaCfc(invdA) |
| | AM02656-AS | 139 | usGfsgUfcUfuUfcAfcuuUfcUfuGfgGfcsuscuAu |
| AD02068 | AM03183-SS | 140 | (Alk-C6-C6)(Alk-C6-Ser)(Alk-C6-Ser)(Alk-C6-Ser)uAuAuGfscsCfcAfaG |
| | | | faAfAfGfuGfaAfaGfaCfc(invdA) |
| | AM03157-AS | 141 | usGfsgucuuUfcAfcuuUfcuugggcsuscuAu |
| AD02765 | AM03571-SS | 142 | (Alk-C6-C6)uuAgagscsccaagaAfaGfugaaagacc(invdA) |
| | AM03157-AS | 143 | usGfsgucuuUfcAfcuuUfcuugggcsuscuAu |
| AD02766 | AM03573-SS | 144 | (Alk-C6-C6)uuAuugscsccaagaAfaGfugaaagacc(invdA) |
| | AM03157-AS | 145 | usGfsgucuuUfcAfcuuUfcuugggcsuscuAu |
| AD02767 | AM03575-SS | 146 | (Alk-C6-C6)uuGAugscsccaagaAfaGfugaaagacc(invdA) |
| | AM03157-AS | 147 | usGfsgucuuUfcAfcuuUfcuugggcsuscuAu |
| AD02769 | AM03579-SS | 148 | (Alk-C6-C6)uAugagscsccaagaAfaGfugaaagacc(invdA) |
| | AM02507-AS | 149 | dTsGfgUfcUfuUfcAfcUfuUfCfuuGfgGfcucuAu |
| AD02772 | AM03586-SS | 150 | (Chol-TEG)aUaGasGfcCfcAfAfgaAfaGfuGfaAfaGfaCfc(invdA) |
| | AM02657-AS | 151 | usGfsaAfgCfuGfaGfgCfuCfaAfaGfcAfcUfsusuAu |
| AD01610 | AM02655-SS | 152 | uAuAusGfsuGfcUfuUfgAfgCfcUfcAfgCfuUfcAf(C11-PEG3-NAG3) |
| | AM02474-AS | 153 | dTsGfsaAfgCfuGfaGfgCfuCfaAfaGfcAfcUfsusuAu |
| AD01775 | AM02867-SS | 154 | (Spermine)uAuAusGfsuGfcUfuUfgAfgCfcUfcAfgCfuUfcAf(C11-PEG3-NAG3) |
| | AM02967-AS | 155 | usGfsgucUfuucAfcuuUfcUfugggcsuscuAu |
| AD01856 | AM02960-SS | 156 | uAuAugscsccaagaaAfGfugaaagacca(C11-PEG3-NAG3) |
| | AM03109-AS | 157 | usGfsgUfcUfuUfcAfcuuUfcUfuGfgGfcsTMsCMuAu |
| AD01975 | AM03112-SS | 158 | uAuAuGfscsCfcAfaGfaAfAfGfuGfaAfaGfaCfCMAM(C11-PEG3-NAG3) |
| | AM02656-AS | 159 | usGfsgUfcUfuUfcAfcuuUfcUfuGfgGfcsuscuAu |
| AD01994 | AM03137-SS | 160 | uAuAuGfscsCfcAfaGfaAfAfGfuGfaAfaGfauAu(C6-PEG4-NAG3) |
| | AM03410-AS | 161 | uAusGfsgucuuUfcAfcuuUfcuugggcsuscu |
| AD02665 | AM03428-SS | 162 | uAugscsccaagaAfaGfugaaagaccsusAu(NAG13) |
| | AM03410-AS | 163 | uAusGfsgucuuUfcAfcuuUfcuugggcsuscu |
| AD02666 | AM03429-SS | 164 | uAugscsccaagaAfaGfugaaagacc(invdA)Au(NAG13) |
| | AM03410-AS | 165 | uAusGfsgucuuUfcAfcuuUfcuugggcsuscu |
| AD02703 | AM03479-SS | 166 | uAugscsccaagaAfaGfugaaagaccauu(NAG13) |
| | AM03410-AS | 167 | uAusGfsgucuuUfcAfcuuUfcuugggcsuscu |
| AD02704 | AM03480-SS | 168 | uAugscsccaagaAfaGfugaaagaccaua(NAG13) |
| | AM03410-AS | 169 | uAusGfsgucuuUfcAfcuuUfcuugggcsuscu |
| AD02809 | AM03634-SS | 170 | uAugscsccaagaAfaGfugaaagaccuAu(NAG18) |

Exemplary Factor VII RNAi agents and sequences

| | | | |
|---|---|---|---|
| | AM00026-AS | 171 | dTsGfaGfuUfgGfcAfcGfcCfuUfuGfcdTsdT |
| AD01149 | AM01952-SS | 172 | (Alk-C6)uGuGfcAfaAfgGfcGfuGfcCfaAfcUfcAf(invdT) |
| | AM00026-AS | 173 | dTsGfaGfuUfgGfcAfcGfcCfuUfuGfcdTsdT |
| AD01259 | AM02094-SS | 174 | (DBCO-TEG)uGuGfcAfaAfgGfcGfuGfcCfaAfcUfcAf(invdT) |

Exemplary LPA RNAi agents and sequences

| | | | |
|---|---|---|---|
| | AM02412-AS | 175 | dTsGfsaCfaCfcUfgAfuUfcUfgUfuUfcUfgAfsgsuAu |
| AD01466 | AM02445-SS | 176 | uAuAusCfsaGfaAfaCfaGfaAfuCfaGfgUfgUfcAf(C6-SS -Alk-Me) |
| | AM02404-AS | 177 | dTsGfsaGfaAfuGfaGfcCfuCfgAfuAfaCfuCfsusuAu |
| AD01462 | AM02441-SS | 178 | uAuAusAfsgUfuAfuCfgAfgGfcUfcAfuUfcUfcAf(C6-SS -Alk-Me) |
| | AM03427-AS | 179 | dTsGfaGfaAfuGfaGfccuCfgAfuAfaCfuCfuuAu |
| AD02664 | AM03426-SS | 180 | (Chol-TEG)uAuAusAfgUfuAfuCfgAfGfGfcUfcAfuUfcUfc(invdA) |
| | AM02532-AS | 181 | dTsGfscGfuCfuGfaGfcauUfgUfgUfcAfgGfsusuAu |
| AD01530 | AM02538-SS | 182 | uAuAusCfsuGfaCfaCfaAfUfGfcUfcAfgAfcGfcAf(C11-PEG3-NAG3) |
| | AM02533-AS | 183 | dTsUfsgCfgUfcUfgAfgcaUfuGfuGfuCfaGfsgsuAu |
| AD01531 | AM02539-SS | 184 | uAuAusUfsgAfcAfcAfaUfGfCfuCfaGfaCfgCfaAf(C11-PEG3-NAG3) |
| | AM02536-AS | 185 | dTsAfsaGfgGfcGfaAfucuCfaGfcAfuCfuGfsgsuAu |
| AD01534 | AM02542-SS | 186 | uAuAusAfsgAfuGfcUfgAfGfAfuUfcGfcCfcUfuAf(C11-PEG3-NAG3) |
| | AM02534-AS | 187 | dTsGfsaGfaAfuGfaGfccuCfgAfuAfaCfuCfsusuAu |
| AD01532 | AM02540-SS | 188 | uAuAusAfsgUfuAfuCfgAfGfGfcUfcAfuUfcUfcAf(C11-PEG3-NAG3) |
| | AM03119-AS | 189 | usGfsaGfaAfuGfaGfccuCfgAfuAfaCfuCMsTMsuAu |
| AD01981 | AM03122-SS | 190 | uAuAusAfsgUfuAfuCfgAfGfGfcUfcAfuUfcUfCMAM(C11-PEG3-NAG3) |
| | AM02857-AS | 191 | usGfsaGfaAfuGfaGfccuCfgAfuAfaCfuCfsusuAu |
| AD01979 | AM03122-SS | 192 | uAuAusAfsgUfuAfuCfgAfGfGfcUfcAfuUfcUfCMAM(C11-PEG3-NAG3) |
| | AM03255-AS | 193 | usGfsaGfaAfugaGfccuCfgauaaCfuCfsusuAu |
| AD02435 | AM03238-SS | 194 | uAuAusAfsgUfuAfuCfgAfGfGfcUfcauucuca(C11-PEG3-NAG3) |
| | AM03377-AS | 195 | usGfsagaauGfaGfccuCfgauaacucsusuAu |
| AD02619 | AM03243-SS | 196 | uAuAusasguuaucgAfGfGfcucauucuca(C11-PEG3-NAG3) |
| | AM02535-AS | 197 | dTsGfsaCfaCfcUfgAfuucUfgUfuUfcUfgAfsgsuAu |
| AD01533 | AM02541-SS | 198 | uAuAusCfsaGfaAfaCfaGfAfAfuCfaGfgUfgUfcAf(C11-PEG3-NAG3) |
| | AM02863-AS | 199 | usGfsaCfaCfcUfgAfuucUfgUfuUfcUfgAfsgsuAu |
| AD01772 | AM02541-SS | 200 | uAuAusCfsaGfaAfaCfaGfAfAfuCfaGfgUfgUfcAf(C11-PEG3-NAG3) |
| | AM02864-AS | 201 | usgsaCfaCfcUfgAfuucUfgUfuUfcUfgAfsgsuAu |
| AD01773 | AM02541-SS | 202 | uAuAusCfsaGfaAfaCfaGfAfAfuCfaGfgUfgUfcAf(C11-PEG3-NAG3) |
| | AM02865-AS | 203 | usgsaCfaCfcUfgAfuucUfgUfuUfcUfgasgsuAu |
| AD01774 | AM02541-SS | 204 | uAuAusCfsaGfaAfaCfaGfAfAfuCfaGfgUfgUfcAf(C11-PEG3-NAG3) |
| | AM03490-AS | 205 | vpdTCfsgUfaUfaAfcAfauaAfgGfgGfcUfgCfscsuAu |
| AD02714 | AM03492-SS | 206 | uAuAusCfsaGfcCfcCfuUfAfUfuGfuUfaUfaCfga(NAG13) |
| | AM03107-AS | 207 | usCfsgUfaUfaAfcAfauaAfgGfgGfcUfgCfscsuAu |
| AD02552 | AM03289-SS | 208 | uAuAuscsagccccuUfAfUfuguuauacga(C11-PEG3-NAG3) |
| | AM03283-AS | 209 | usCfsgUfaUfaacaauaAfgGfgGfcugcscsuAu |
| AD02752 | AM03546-SS | 210 | uAuAuscsagccccuUfAfUfuguuauacga(NAG13) |

Exemplary Hif2alpha RNAi agents and sequences

| | | | |
|---|---|---|---|
| | AM02145-AS | 211 | dTsUfsuCfaUfgAfaAfucgUfuAfcGfuUfggscsuGu |
| AD01293 | AM02149-SS | 212 | uAuAusCfsaAfcGfuAfaCfGfAfuUfuCfaUfgAfaAf(C6-SS-Alk-Me) |
| | AM02146-AS | 213 | dTsUfsuCfaUfgAfaAfucgUfuAfcGfuCfggscsuGu |
| AD01294 | AM02163-SS | 214 | uAuAusCfsgAfcGfuAfaCfGfAfuUfuCfaUfgAfaAf(C6-SS-Alk-Me) |
| | AM02147-AS | 215 | dTsUfsuCfaUfgAfaAfucgUfuAfcGfuUfggscsuAu |
| AD01295 | AM02149-SS | 216 | uAuAusCfsaAfcGfuAfaCfGfAfuUfuCfaUfgAfaAf(C6-SS-Alk-Me) |
| | AM02150-AS | 217 | dTsUfsuCfaUfgAfaAfucgUfuAfcGfuUfggcusdTsdT |
| AD01296 | AM02149-SS | 218 | uAuAusCfsaAfcGfuAfaCfGfAfuUfuCfaUfgAfaAf(C6-SS-Alk-Me) |
| | AM02346-AS | 219 | dTsUfuCfaUfgAfAUNAAfuCfgUfuAfcGfuCfggcsuAu |
| AD01411 | AM02365-SS | 220 | uAuAusCfgAfcGfuAfaCfgAfuUfuCfaUfgAfaAf(C6-SS -Alk-Me) |

### Example 2

### In Vivo Analysis of 26Mer Factor XII (F12) RNAi Agent Efficacy In Vivo

### A) Administration and Sample Collection.

In order to evaluate the efficacy of 26mer F12 RNAi agents in vivo, wild-type mice were used. For some experiments, cholesterol-conjugated 26mer F12 RNAi agents were administered to mice using MLP delivery polymer on day 1. Each mouse received an intravenous (IV) injection into the tail vein of 200-250 μL solution containing a dose of RNAi agent+MLP delivery polymer (1:1 w/w RNAi agent: MLP delivery polymer in most cases). For other experiments, the indicated 26mer F12 RNAi agent was administered by subcutaneous injection. Control serum (pre-treatment) samples were taken from the mice pre-injection on days −7, −5, −4, or −1. Post injection serum samples were taken from the mice days 4, 8, 15, 22, 29, 36, 43, 50, 53, 57, 64, and/or 71.

### B) Factor 12 Serum Protein Levels.

F12 protein (mF12) levels in serum were monitored by assaying serum from the mice using an ELISA for mouse F12 (Molecular Innovations) until mF12 expression levels returned to baseline. For normalization, mF12 level for each animal at a time point was divided by the pre-treatment level of expression in that animal to determine the ratio of expression “normalized to pre-treatment”. Expression at a specific time point was then normalized to the saline control group by dividing the “normalized to day pre-treatment” ratio for an individual animal by the mean “normalized to day pre-treatment” ratio of all mice in the saline control group. This resulted in expression for each time point normalized to that in the control group. Experimental error is given as standard deviation.

TABLE 3
Serum F12 protein levels in wild-type mice following
administration of 26mer F12 RNAi agents. Cholesterol-
onjugated 26mer F12 RNAi agents were administered
to mice using MLP delivery polymer.

| | | Delivery | |
|---|---|---|---|
| Duplex ID | RNAi agent | Polymer | Relative F12 |
| no. | (mg/kg) | (mg/kg) | levels |
| AD01457 | 2 | 2 | 0.088 |
| AD01459 | 2 | 2 | 0.197 |
| AD01520 | 2 | 2 | 0.012 |
| AD01537 | 10 | 10 | 0.588 |
| AD01538 | 10 | 10 | 0.705 |
| AD01539 | 10 | 10 | 0.788 |
| AD01540 | 10 | 10 | 0.661 |
| AD01541 | 10 | 10 | 0.577 |
| AD01542 | 10 | 10 | 0.470 |
| AD01543 | 10 | 10 | 0.774 |
| AD01544 | 10 | 10 | 0.647 |
| AD01545 | 10 | 10 | 0.820 |
| AD01577 | 2 | 2 | 0.790 |
| AD01579 | 2 | 2 | 0.538 |
| AD02068 | 5 | 5 | 0.038 |
| AD02765 | 0.4 | 4 | 0.010 |
| AD02766 | 0.4 | 4 | 0.013 |
| AD02767 | 0.4 | 4 | 0.014 |
| AD02769 | 0.4 | 4 | 0.014 |
| AD02772 | 2 | 2 | 0.700 |

TABLE 4
Serum F12 protein levels in wild-type mice following administration
of 26mer F12 RNAi agents. 26mer F12 RNAi agents were
administered to mice by subcutaneous injection.

| | RNAi agent | |
|---|---|---|
| Duplex ID No. | (mg/kg) | F12 activity |
| AD01610 | 10 | 0.637 |
| AD01775 | 10 | 0.660 |
| AD01856 | 10 | 0.034 |
| AD01975 | 5 | 0.444 |
| AD01994 | 5 | 0.903 |
| AD02665 | 3 | 0.241 |
| AD02666 | 3 | 0.151 |
| AD02703 | 3 | 0.308 |
| AD02704 | 3 | 0.288 |
| AD02809 | 3 | 0.610 |

### Example 3

### In Vivo Analysis of 26Mer Factor VII RNAi Agent Efficacy In Vivo

A) 120 μg polyacrylate polymer (1095-126) was modified with 2×AC-NAG and 6×AC-PEG12. The modified polymer was then conjugated to 12 μg of AD-01149 26mer FVII RNAi agent and administered to ICR mice by subcutaneous injection. Samples were collected on day 5 and assayed for Factor VII.

TABLE 5
Relative Factor VII expression following
administration of 26mer FVII RNAi agent

| | Relative Factor VII |
|---|---|
| Treatment | expression |
| isotonic glucose | 1 ± 0.06 |
| AD-01149 | 0.65 ± 0.18 |

B) 20 μg MLP was modified with 2×CDM-NAG followed by 3×CDM-NAG. The modified MLP was combined with 30 μg of AD-01259 26mer FVII RNAi agent and administered to ICR mice by intravascular injection. Samples were collected on day 5 and assayed for Factor VII.

TABLE 6
Relative Factor VII expression following
administration of 26mer FVII RNAi agent.

| Treatment | Relative expression |
|---|---|
| isotonic glucose | 1 ± 0.34 |
| AD-01259 | 0.12 ± 0.05 |

### Example 4

### In Vivo Analysis of 26Mer Hif2α RNAi Agent Efficacy In Vivo

RGD Targeted HiF2α-RNAi agent delivery conjugates were formed using RGD mimic-PEG-HyNic masking. 400 μg 126 or 100 A polymer was modified with 8×PEG₁₂-ACit-PABC-PNP/0.5× aldehyde-PEG₂₄-FCit-PABC-PNP (with RGD mimic #1-PEG-HyNic using protocol #1) (WO 2012/092373 and WO 2015/021092) and 80 μg of the indicated Hif2α RNAi agent. Kidney RCC tumor-bearing mice were generated as described and treated with a single tail vein injection of isotonic glucose or the indicated Hif2α-ITG-DPC (Hif2α-ITG-DPC=Hif2α RNAi agent-delivery polymer conjugate. The delivery polymer was modified with RGD ligand and PEG masking agents). Mice were euthanized 72 h after injection and total RNA was prepared from kidney tumor using Trizol reagent following manufacture's recommendation. Relative HiF2α mRNA levels were determined by RT-qPCR as described below and compared to mice treated with delivery buffer (isotonic glucose) only.

TABLE 7
Hif2α knockdown in mice following Hif2α RNAi agent
delivery. RNAi agents were conjugated to the indicated
reversibly masked delivery polymer.

| | | | | Relative Expression | |
|---|---|---|---|---|---|
| RNAi agent | | Polymer | | | low error/ |
| duplex number | μg | number | μg | day 4 | high error |
| isotonic glucose | 0 | | 0 | 1.00 | 0.06/0.06 |
| AD01293 | 80 | 126 | 400 | 0.20 | 0.01/0.01 |
| AD01294 | 80 | 126 | 400 | 0.17 | 0.01/0.02 |
| AD01295 | 80 | 126 | 400 | 0.22 | 0.02/0.02 |
| AD01296 | 80 | 126 | 400 | 0.21 | 0.04/0.06 |
| AD01411 | 150 | 100A | 300 | 0.36 | 0.01/0.01 |

### Quantitative Real-Time PCR Assay.

In preparation for quantitative PCR, total RNA was isolated from tissue samples homogenized in TriReagent (Molecular Research Center, Cincinnati, Ohio) following the manufacturer's protocol. Approximately 500 ng RNA was reverse-transcribed using the High Capacity cDNA Reverse Transcription Kit (Life Technologies). For human (tumor) Hif2α (EPAS1) expression, pre-manufactured TaqMan gene expression assays for human Hif2α (Catalog #4331182) and CycA (PPIA) Catalog #: 4326316E) were used in biplex reactions in triplicate using TaqMan Gene Expression Master Mix (Life Technologies) or VeriQuest Probe Master Mix (Affymetrix). For human (tumor) VegFa (VEGFA) expression, pre-manufactured TaqMan gene expression assays for human VegFa (Catalog #4331182, Assay ID: Hs00900055) and CycA (Part#: 4326316E) were used in biplex reactions in triplicate using TaqMan Gene Expression Master Mix (Life Technologies) or VeriQuest Probe Master Mix (Affymetrix). Quantitative PCR was performed by using a 7500 Fast or StepOnePlus Real-Time PCR system (Life Technologies). The ΔΔC_{T} method was used to calculate relative gene expression.

Polymer APN 1095-126 (126): propyl acrylate/ethoxyethylamine acrylate membrane active amphipathic copolymer.

| MW | Theoretical MW | | % Amine | % Alkyl | % End Group | Azides Per |
|---|---|---|---|---|---|---|
| (protected) | (deprotected) | PDI | Incorporation | Incorporation | Removal | Polymer |
| 66,670 | 47,606 | 1.11 | 56 | 44 | 0 | 4.1 |

Polymer APN 1170-100 A (100 A) propyl acrylate/ethoxyethylamine acrylate membrane active amphipathic copolymer.

| | MW | Theoretical MW | | % Amine | % Alkyl | % End Group | Azides/ |
|---|---|---|---|---|---|---|---|
| Polymer | (protected) | (deprotected) | PDI | Incorp. | Incorp. | Removal | Polymer |
| APN 1170-100A | 64,430 | 45,765 | 1.22 | 56 | 44 | 0 | 1.25 |

### Protocol 1.

The indicated polymer was reacted with SMPT at a weight ratio of 1:0.015 (polymer: SMPT) in 5 mM HEPES, pH 8.0 buffer for 1 h at RT. The SMPT-modified polymer was then reacted with aldehyde-PEG-dipeptide masking agent (aldehyde-PEG₁₂-FCit or aldehyde-PEG₂₄-ACit) at desired ratios for 1 h at RT. The modified polymer was then reacted with PEG₁₂-dipeptide masking agent (PEG₁₂-FCit, PEG₁₂-ACit or PEG₂₄-ACit) at a weight ratio of 1:2 (polymer:PEG) in 100 mM HEPES, pH 9.0 buffer for 1 h at RT. The modified polymer was then reacted overnight with SATA-RNAi agent at a weight ratio of 1:0.2 (polymer:SATA-RNAi agent) in 100 mM HEPES, pH 9.0 buffer at RT to attach the RNAi agent. Next, the modified polymer was reacted with protease cleavable PEG (PEG12-FCit or PEG12-ACit or PEG24-ACit) at a weight ratio of 1:6 (polymer:PEG) in 100 mM HEPES, pH 9.0 buffer for 1 h at RT. The resultant conjugate was purified using a sephadex G-50 spin column.

RGD-HyNic (Example 6B) was attached to the modified polymer to form the full delivery conjugate by reaction with the modified polymer at a weight ratio of 1:0.7 (polymer:RGD-HyNic mimic) in 50 mM MES, pH 5.0 buffer for a minimum of 4 h at RT. The conjugate was purified using a sephadex G-50 spin column. RGD ligand attachment efficiency was determined as described above.

### Example 5

### In Vivo Analysis of 26Mer LPA RNAi Agent Efficacy In Vivo

For some experiments, a plasmid containing LPA target sequences inserted into the 3′ UTR of secreted placental alkaline phosphatase (SEAP) was injected into wild-type mice by hydrodynamic tail vein injection. At four to five weeks post HTV injection, RNAi agents were administered to these transiently transgenic SEAP-LPA HTV mice.

For other experiments, apo(a) and Lp(a) transgenic mice (Frazer K A et al 1995, Nature Genetics 9:424-431) were used. The apo(a) transgenic mice expresses human apo(a) from a YAC containing the full LPA gene (encoding apo(a) protein) with additional sequences both 5′ and 3′. Lp(a) mice were bred by crossing apo(a) YAC-containing mice to human apoB-100 expressing mice (Callow M J et al 1994, PNAS 91:2130-2134, Lawn R M et al. 1992 Nature 360(6405): 670-672).

### A) Intravascular Administration of 26Mer LPA RNAi Agent:

Polymer ARF1164-106A-5 was masked with AC-NAG and AC-PEG12 and conjugated to the 26mer LPA RNAi agent. Each mouse received an intravenous (IV) injection into the tail vein of 200-250 μL solution containing a dose of 26mer LAP RNAi agent attached to protease-masked polymer. Control serum (pre-treatment) samples were taken from the mice pre-injection on day −1. Post injection serum samples were taken from the mice on various days. Polymer ARF1164-106A-5 is a propyl acrylate and ethyl ethoxy amino acrylate (54%) copolymer having a PDI of 1.043.

### B) Subcutaneous Administration of 26Mer LPA RNAi Agent:

The indicated 26mer LPA RNAi agent was administered by subcutaneous injection of 100 μl to 300 μl RNAi agent in buffer into the loose skin on the back between the shoulders.

### C) Target Gene Knockdown Evaluation.

SEAP protein (SEAP) levels in serum were monitored by assaying serum from the mice using a chemiluminescent substrate (Tropix® Phospha-Light™, Applied Biosystems) until SEAP levels returned to baseline. For normalization, the SEAP level for each animal at a time point was divided by the pre-treatment level of expression in that animal to determine the ratio of expression “normalized to pre-treatment”. Expression at a specific time point was then normalized to the saline control group by dividing the “normalized to day pre-treatment” ratio for an individual animal by the mean “normalized to day pre-treatment” ratio of all mice in the saline control group. This resulted in expression for each time point normalized to that in the control group. Experimental error is given as standard deviation. For LP(a) transgenic mice, Apo(a) levels were measured by ELISA and LP(a) levels were measured by clinical chemistry analyzer (Cobas). A decrease in target gene expression was observed following administration of all the 26mer LPA RNAi agents tested.

TABLE 8
Target gene knockdown in mice following administration of 26mer
LPA RNAi agents. Cholesterol-conjugated 26mer LPA RNAi agents
were administered to mice using MLP delivery polymer.

| | RNAi agent | Delivery Polymer | Target gene |
|---|---|---|---|
| Duplex ID No. | (mg/kg) | (mg/kg) | knockdown |
| AD01466 | 0.5 | 2 | 0.329 |
| AD01462 | 0.5 | 2 | 0.319 |
| AD02664 | 2 | 2 | 0.001 |
| AD01530 | 1 | 2 | 0.264 |
| AD01531 | 1 | 2 | 0.220 |

TABLE 9
Target gene knockdown in mice following administration
of 26mer LPA RNAi agents. 26mer LPA RNAi agents were
administered to mice by subcutaneous injection.

| | RNAi agent | Target gene |
|---|---|---|
| Duplex ID No. | (mg/kg) | knockdown |
| AD01534 | 10 | 0.398 |
| AD01532 | 10 | 0.434 |
| AD01981 | 3 | 0.049 |
| AD01979 | 3 | 0.057 |
| AD02435 | 10 | 0.038 |
| AD02619 | 3 | 0.024 |
| AD01533 | 10 | 0.299 |
| AD01772 | 10 | 0.247 |
| AD01773 | 10 | 0.2759 |
| AD01774 | 10 | 0.370 |
| AD02714 | 3 | 0.064 |
| AD02552 | 10 | 0.033 |
| AD02752 | 3 | 0.081 |

1. A double-stranded RNAi agent capable of inhibiting the expression of a target gene, comprising a sense strand and an antisense strand, wherein each strand has 26 nucleotides, the double-stranded RNAi agent in blunt-ended, a region of at least 85% complementarity over at least 18 consecutive nucleotides, the sense strand contains a 2′-OMethyl uridine at the 5′ terminal positions, the sense strand contains at least one ribonucleotide at the second or third nucleotide from the 5′ end, and both the sense strand and antisense strand contain one or more modified nucleotides.

2. The double-stranded RNAi agent of claim 1 wherein nucleotides 2-19 from the 5′ end of the antisense strand are at least 85% complementary to a sequence in a target mRNA.

3. The double-stranded RNAi agent of claim 2 wherein nucleotides 8-25, 7-25, 6-25, 5-25, 4-25, 9-26, 8-26, 7-26, 6-26, 5-26, or 4-26 from the 5′ end of the sense strand are at least 85%, at least 90%, or 100% complementary to the corresponding sequence in the antisense strand.

4. The double-stranded RNAi agent of claim 1 wherein the sense strand contains a ribonucleotide at the second nucleotide from the 5′ end of the sense strand and all other nucleotides of the sense strand are modified.

5. The double-stranded RNAi agent of claim 4 wherein the sense strand contains ribonucleotides at the second and fourth nucleotides from the 5′ end of the sense strand and all other nucleotide of the sense strand are modified.

6. The double-stranded RNAi agent of claim 1 wherein the sense strand contains ribonucleotides at the third and fourth nucleotides from the 5′ end of the sense strand and all other nucleotides of the sense strand are modified.

7. The double-stranded RNAi agent of claim 4 wherein the first three nucleotides from the 5′ end of the sense strand are, in order, 2′-OMethyl uridine, ribo-adenosine, and 2′-OMethyl uridine.

8. The double-stranded RNAi agent of claim 5 wherein the first four nucleotides from the 5′ end of the sense strand are, in order, 2′-OMethyl uridine, ribo-adenosine, 2′-OMethyl uridine, and ribo-adenosine.

9. The double-stranded RNAi agent of claim 1 wherein the 3′ terminal nucleotide of the antisense strand is a 2′-Fluoro nucleotide, a 2′-OMethyl nucleotide, an inverted nucleotide, a 3′-OMe nucleotide, or a 2′-methoxyethyl nucleotide.

10. The double-stranded RNAi agent of claim 1 wherein the 5′ terminal nucleotide of the antisense strand is a 2′-deoxy nucleotide, a 2′-OMethyl nucleotide, an inverted nucleotide, an abasic nucleotide, 3′-OMe nucleotide, or a 2′-methoxyethyl nucleotide.

11. The double-stranded RNAi agent of claim 1 wherein the five nucleotides at the 3′ end of the antisense strand are (5′ to 3′): (2′-OMethyl nucleotide)₅, (2′-OMethyl nucleotide)₃(2′-deoxy nucleotide)₂, (2′-OMethyl nucleotide)₃(inverted 2′-deoxy nucleotide)(2′-OMethyl nucleotide), (2′-OMethyl nucleotide)₃(ribonucleotide)₂, (2′-OMethyl nucleotide)₃(ribonucleotide)₂(2′-OMethyl nucleotide), (2′-OMethyl nucleotide)₃(2′-methoxyethyl nucleotide)₂, (2′-OMethyl nucleotide)(ribonucleotide)₄, (2′-OMethyl nucleotide)(ribonucleotide)(2′-OMethyl nucleotide)(2′-Fluoro nucleotide)(2′-OMethyl nucleotide), (2′-OMethyl nucleotide)₂(2′-Fluoro nucleotide)(2′-OMethyl nucleotide)₂, (2′-Fluoro nucleotide)(2′-OMethyl nucleotide)₂(ribonucleotide)(2′-OMethyl nucleotide), or (2′-methoxyethyl nucleotide)₂(2′-OMethyl nucleotide)(ribonucleotide)(2′-OMethyl nucleotide).

12. The double-stranded RNAi agent of claim 1, wherein the one or more modified nucleotides are selected from the group consisting of: 2′-OMe nucleotide, 2′-Fluoro nucleotide, 2′-deoxy nucleotide, 2′,3′-seco nucleotide, locked nucleotide, 2′-F-Arabino nucleotide, 2′-methoxyethyl nucleotide, abasic ribose, ribitol, inverted nucleotide, inverted abasic nucleotide, inverted 2′-OMe nucleotide, inverted 2′-deoxy nucleotide, 2′-amino-modified nucleotide, 2′-alkyl-modified nucleotide, morpholino nucleotide, vinyl phosphonate deoxyribonucleotide, 3′-OMe nucleotide.

13. The double-stranded RNAi agent of claim 1 wherein 20% or fewer of the modified nucleotides are 2′-Fluoro modified nucleotides

14. The double-stranded RNAi agent of claim 1, wherein the double-stranded RNAi agent contains at least one phosphorothioate internucleotide linkage.

15. The double-stranded RNAi agent of claim 1, wherein the double-stranded RNAi agent contains at least two phosphorothioate internucleotide linkages.

16. The double-stranded RNAi agent of claim 1, wherein the double-stranded RNAi agent contains at least four phosphorothioate internucleotide linkages.

17. The double-stranded RNAi agent of claim 1, wherein the double-stranded RNAi agent contains at least six phosphorothioate internucleotide linkages.

18. The double-stranded RNAi agent of claim 1 wherein the double-stranded RNAi agent is covalently linked to a targeting group.

19. The double-stranded RNAi agent of claim 18 wherein the targeting group is covalently linked to the sense strand.

20. The double-stranded RNAi agent of claim 19 wherein the targeting group is covalently linked to the 5′ end of the sense strand.

21. The double-stranded RNAi agent of claim 19 wherein the targeting group is selected from the group consisting of: NAG3, NAG3, NAG14, NAG15, NAG16, NAG17, NAG18, NAG19, NAG20, and NAG 21.

22. The double-stranded RNAi agent of claim 19 wherein the targeting group comprises a cholesterol or a cholesteryl derivative.

23. The double-stranded RNAi agent of claim 22 wherein cholesterol or a cholesteryl derivative is linked to the double-stranded RNAi via a linker.

24. The double-stranded RNAi agent of claim 1 wherein the double-stranded RNAi agent is covalently linked to a delivery polymer.

25. A method for inhibiting the expression of a gene in vivo, comprising: (a) introducing into the cell a double-stranded RNAi agent of claim 1.


SEQUENCE LISTING (220 sequences)

SEQ ID NO: 1 - DNA, 26 bp, artificial sequence
  RNAi agent strand
tgagaagcugaggcucaaagcacuau

SEQ ID NO: 2 - DNA, 26 bp, artificial sequence
  RNAi agent strand
tgagaagcugaggcucaaagcauaua

SEQ ID NO: 3 - DNA, 26 bp, artificial sequence
  RNAi agent strand
tggucuuucacuuucuugggcucuau

SEQ ID NO: 4 - DNA, 26 bp, artificial sequence
  RNAi agent strand
tcacuuucuugggcuccaaacaguau

SEQ ID NO: 5 - DNA, 26 bp, artificial sequence
  RNAi agent strand
tucacuuucuugggcuccaaacauau

SEQ ID NO: 6 - DNA, 26 bp, artificial sequence
  RNAi agent strand
tuucacuuucuugggcuccaaacuau

SEQ ID NO: 7 - DNA, 26 bp, artificial sequence
  RNAi agent strand
tuuucacuuucuugggcuccaaauau

SEQ ID NO: 8 - DNA, 26 bp, artificial sequence
  RNAi agent strand
tagcugaggcucaaagcacuucuuau

SEQ ID NO: 9 - DNA, 26 bp, artificial sequence
  RNAi agent strand
tgaagcugaggcucaaagcacuuuau

SEQ ID NO: 10 - DNA, 26 bp, artificial sequence
  RNAi agent strand
tuuguugcggucaccacagcccguau

SEQ ID NO: 11 - DNA, 26 bp, artificial sequence
  RNAi agent strand
tgcuuguugcggucaccacagccuau

SEQ ID NO: 12 - DNA, 26 bp, artificial sequence
  RNAi agent strand
tggcuuguugcggucaccacagcuau

SEQ ID NO: 13 - DNA, 26 bp, artificial sequence
  RNAi agent strand
tggucuuucacuuucuugggcucuau

SEQ ID NO: 14 - DNA, 26 bp, artificial sequence
  RNAi agent strand
tggucuuucacuuucuugggcucuau

SEQ ID NO: 15 - RNA, 26 nt, artificial sequence
  RNAi agent strand
uggucuuucacuuucuugggcucuau

SEQ ID NO: 16 - RNA, 26 nt, artificial sequence
  RNAi agent strand
uggucuuucacuuucuugggcucuau

SEQ ID NO: 17 - RNA, 26 nt, artificial sequence
  RNAi agent strand
uggucuuucacuuucuugggcucuau

SEQ ID NO: 18 - RNA, 26 nt, artificial sequence
  RNAi agent strand
uggucuuucacuuucuugggcucuau

SEQ ID NO: 19 - RNA, 26 nt, artificial sequence
  RNAi agent strand
uggucuuucacuuucuugggcucuau

SEQ ID NO: 20 - DNA, 26 bp, artificial sequence
  RNAi agent strand
tggucuuucacuuucuugggcucuau

SEQ ID NO: 21 - RNA, 26 nt, artificial sequence
  RNAi agent strand
ugaagcugaggcucaaagcacuuuau

SEQ ID NO: 22 - DNA, 26 bp, artificial sequence
  RNAi agent strand
tgaagcugaggcucaaagcacuuuau

SEQ ID NO: 23 - RNA, 26 nt, artificial sequence
  RNAi agent strand
uggucuuucacuuucuugggcucuau

SEQ ID NO: 24 - DNA, 26 bp, artificial sequence
  RNAi agent strand
uggucuuucacuuucuugggctcuau

SEQ ID NO: 25 - RNA, 26 nt, artificial sequence
  RNAi agent strand
uggucuuucacuuucuugggcucuau

SEQ ID NO: 26 - RNA, 26 nt, artificial sequence
  RNAi agent strand
uauggucuuucacuuucuugggcucu

SEQ ID NO: 27 - RNA, 26 nt, artificial sequence
  RNAi agent strand
uauggucuuucacuuucuugggcucu

SEQ ID NO: 28 - RNA, 26 nt, artificial sequence
  RNAi agent strand
uauggucuuucacuuucuugggcucu

SEQ ID NO: 29 - RNA, 26 nt, artificial sequence
  RNAi agent strand
uauggucuuucacuuucuugggcucu

SEQ ID NO: 30 - RNA, 26 nt, artificial sequence
  RNAi agent strand
uauggucuuucacuuucuugggcucu

SEQ ID NO: 31 - RNA, 26 nt, artificial sequence
  RNAi agent strand
uauaugcuuugagccucagcuucuca

SEQ ID NO: 32 - RNA, 26 nt, artificial sequence
  RNAi agent strand
uauaugcuuugagccucagcuucuca

SEQ ID NO: 33 - RNA, 26 nt, artificial sequence
  RNAi agent strand
uauaugcccaagaaagugaaagacca

SEQ ID NO: 34 - RNA, 26 nt, artificial sequence
  RNAi agent strand
uauauguuuggagcccaagaaaguga

SEQ ID NO: 35 - RNA, 26 nt, artificial sequence
  RNAi agent strand
uauauuuuggagcccaagaaagugaa

SEQ ID NO: 36 - RNA, 26 nt, artificial sequence
  RNAi agent strand
uauauuuggagcccaagaaagugaaa

SEQ ID NO: 37 - RNA, 26 nt, artificial sequence
  RNAi agent strand
uauauuggagcccaagaaagugaaaa

SEQ ID NO: 38 - RNA, 26 nt, artificial sequence
  RNAi agent strand
uauauaagugcuuugagccucagcua

SEQ ID NO: 39 - RNA, 26 nt, artificial sequence
  RNAi agent strand
uauaugugcuuugagccucagcuuca

SEQ ID NO: 40 - RNA, 26 nt, artificial sequence
  RNAi agent strand
uauauggcuguggugaccgcaacaaa

SEQ ID NO: 41 - RNA, 26 nt, artificial sequence
  RNAi agent strand
uauaucuguggugaccgcaacaagca

SEQ ID NO: 42 - RNA, 26 nt, artificial sequence
  RNAi agent strand
uauauuguggugaccgcaacaagcca

SEQ ID NO: 43 - RNA, 26 nt, artificial sequence
  RNAi agent strand
uauuagcccaagaaagugaaagacca

SEQ ID NO: 44 - RNA, 26 nt, artificial sequence
  RNAi agent strand
uauaagcccaagaaagugaaagacca

SEQ ID NO: 45 - RNA, 26 nt, artificial sequence
  RNAi agent strand
uauaugcccaagaaagugaaagacca

SEQ ID NO: 46 - RNA, 26 nt, artificial sequence
  RNAi agent strand
uuagagcccaagaaagugaaagacca

SEQ ID NO: 47 - RNA, 26 nt, artificial sequence
  RNAi agent strand
uuauugcccaagaaagugaaagacca

SEQ ID NO: 48 - RNA, 26 nt, artificial sequence
  RNAi agent strand
uugaugcccaagaaagugaaagacca

SEQ ID NO: 49 - RNA, 26 nt, artificial sequence
  RNAi agent strand
uaugagcccaagaaagugaaagacca

SEQ ID NO: 50 - RNA, 26 nt, artificial sequence
  RNAi agent strand
auagagcccaagaaagugaaagacca

SEQ ID NO: 51 - RNA, 26 nt, artificial sequence
  RNAi agent strand
uauaugugcuuugagccucagcuuca

SEQ ID NO: 52 - RNA, 26 nt, artificial sequence
  RNAi agent strand
uauaugugcuuugagccucagcuuca

SEQ ID NO: 53 - RNA, 26 nt, artificial sequence
  RNAi agent strand
uauaugcccaagaaagugaaagacca

SEQ ID NO: 54 - RNA, 26 nt, artificial sequence
  RNAi agent strand
uauaugcccaagaaagugaaagacca

SEQ ID NO: 55 - RNA, 26 nt, artificial sequence
  RNAi agent strand
uauaugcccaagaaagugaaagauau

SEQ ID NO: 56 - RNA, 26 nt, artificial sequence
  RNAi agent strand
uaugcccaagaaagugaaagaccuau

SEQ ID NO: 57 - RNA, 26 nt, artificial sequence
  RNAi agent strand
uaugcccaagaaagugaaagaccaau

SEQ ID NO: 58 - RNA, 26 nt, artificial sequence
  RNAi agent strand
uaugcccaagaaagugaaagaccauu

SEQ ID NO: 59 - RNA, 26 nt, artificial sequence
  RNAi agent strand
uaugcccaagaaagugaaagaccaua

SEQ ID NO: 60 - RNA, 26 nt, artificial sequence
  RNAi agent strand
uaugcccaagaaagugaaagaccuau

SEQ ID NO: 61 - DNA, 26 bp, artificial sequence
  RNAi agent strand
tgacaccugauucuguuucugaguau

SEQ ID NO: 62 - DNA, 26 bp, artificial sequence
  RNAi agent strand
tgagaaugagccucgauaacucuuau

SEQ ID NO: 63 - DNA, 26 bp, artificial sequence
  RNAi agent strand
tgagaaugagccucgauaacucuuau

SEQ ID NO: 64 - DNA, 26 bp, artificial sequence
  RNAi agent strand
tgcgucugagcauugugucagguuau

SEQ ID NO: 65 - DNA, 26 bp, artificial sequence
  RNAi agent strand
tugcgucugagcauugugucagguau

SEQ ID NO: 66 - DNA, 26 bp, artificial sequence
  RNAi agent strand
taagggcgaaucucagcaucugguau

SEQ ID NO: 67 - DNA, 26 bp, artificial sequence
  RNAi agent strand
tgagaaugagccucgauaacucuuau

SEQ ID NO: 68 - DNA, 26 bp, artificial sequence
  RNAi agent strand
ugagaaugagccucgauaacuctuau

SEQ ID NO: 69 - RNA, 26 nt, artificial sequence
  RNAi agent strand
ugagaaugagccucgauaacucuuau

SEQ ID NO: 70 - RNA, 26 nt, artificial sequence
  RNAi agent strand
ugagaaugagccucgauaacucuuau

SEQ ID NO: 71 - RNA, 26 nt, artificial sequence
  RNAi agent strand
ugagaaugagccucgauaacucuuau

SEQ ID NO: 72 - DNA, 26 bp, artificial sequence
  RNAi agent strand
tgacaccugauucuguuucugaguau

SEQ ID NO: 73 - RNA, 26 nt, artificial sequence
  RNAi agent strand
ugacaccugauucuguuucugaguau

SEQ ID NO: 74 - RNA, 26 nt, artificial sequence
  RNAi agent strand
ugacaccugauucuguuucugaguau

SEQ ID NO: 75 - RNA, 26 nt, artificial sequence
  RNAi agent strand
ugacaccugauucuguuucugaguau

SEQ ID NO: 76 - DNA, 26 bp, artificial sequence
  RNAi agent strand
tcguauaacaauaaggggcugccuau

SEQ ID NO: 77 - RNA, 26 nt, artificial sequence
  RNAi agent strand
ucguauaacaauaaggggcugccuau

SEQ ID NO: 78 - RNA, 26 nt, artificial sequence
  RNAi agent strand
ucguauaacaauaaggggcugccuau

SEQ ID NO: 79 - RNA, 26 nt, artificial sequence
  RNAi agent strand
uauaucagaaacagaaucagguguca

SEQ ID NO: 80 - RNA, 26 nt, artificial sequence
  RNAi agent strand
uauauaguuaucgaggcucauucuca

SEQ ID NO: 81 - RNA, 26 nt, artificial sequence
  RNAi agent strand
uauauaguuaucgaggcucauucuca

SEQ ID NO: 82 - RNA, 26 nt, artificial sequence
  RNAi agent strand
uauaucugacacaaugcucagacgca

SEQ ID NO: 83 - RNA, 26 nt, artificial sequence
  RNAi agent strand
uauauugacacaaugcucagacgcaa

SEQ ID NO: 84 - RNA, 26 nt, artificial sequence
  RNAi agent strand
uauauagaugcugagauucgcccuua

SEQ ID NO: 85 - RNA, 26 nt, artificial sequence
  RNAi agent strand
uauauaguuaucgaggcucauucuca

SEQ ID NO: 86 - RNA, 26 nt, artificial sequence
  RNAi agent strand
uauauaguuaucgaggcucauucuca

SEQ ID NO: 87 - RNA, 26 nt, artificial sequence
  RNAi agent strand
uauauaguuaucgaggcucauucuca

SEQ ID NO: 88 - RNA, 26 nt, artificial sequence
  RNAi agent strand
uauauaguuaucgaggcucauucuca

SEQ ID NO: 89 - RNA, 26 nt, artificial sequence
  RNAi agent strand
uauauaguuaucgaggcucauucuca

SEQ ID NO: 90 - RNA, 26 nt, artificial sequence
  RNAi agent strand
uauaucagaaacagaaucagguguca

SEQ ID NO: 91 - RNA, 26 nt, artificial sequence
  RNAi agent strand
uauaucagaaacagaaucagguguca

SEQ ID NO: 92 - RNA, 26 nt, artificial sequence
  RNAi agent strand
uauaucagaaacagaaucagguguca

SEQ ID NO: 93 - RNA, 26 nt, artificial sequence
  RNAi agent strand
uauaucagaaacagaaucagguguca

SEQ ID NO: 94 - RNA, 26 nt, artificial sequence
  RNAi agent strand
uauaucagccccuuauuguuauacga

SEQ ID NO: 95 - RNA, 26 nt, artificial sequence
  RNAi agent strand
uauaucagccccuuauuguuauacga

SEQ ID NO: 96 - RNA, 26 nt, artificial sequence
  RNAi agent strand
uauaucagccccuuauuguuauacga

SEQ ID NO: 97 - DNA, 26 bp, artificial sequence
  RNAi agent strand
tuucaugaaaucguuacguuggcuau

SEQ ID NO: 98 - DNA, 26 bp, artificial sequence
  RNAi agent strand
tuucaugaaaucguuacguuggcugu

SEQ ID NO: 99 - DNA, 26 bp, artificial sequence
  RNAi agent strand
tuucaugaaaucguuacguuggcutt

SEQ ID NO: 100 - DNA, 26 bp, artificial sequence
  RNAi agent strand
tuucaugaaaucguuacgucggcuau

SEQ ID NO: 101 - DNA, 26 bp, artificial sequence
  RNAi agent strand
tuucaugaaaucguuacgucggcugu

SEQ ID NO: 102 - RNA, 26 nt, artificial sequence
  RNAi agent strand
uauaucaacguaacgauuucaugaaa

SEQ ID NO: 103 - RNA, 26 nt, artificial sequence
  RNAi agent strand
uauaucaacguaacgauuucaugaaa

SEQ ID NO: 104 - RNA, 26 nt, artificial sequence
  RNAi agent strand
uauaucaacguaacgauuucaugaaa

SEQ ID NO: 105 - RNA, 26 nt, artificial sequence
  RNAi agent strand
uauaucgacguaacgauuucaugaaa

SEQ ID NO: 106 - RNA, 26 nt, artificial sequence
  RNAi agent strand
uauaucgacguaacgauuucaugaaa

SEQ ID NO: 107 - DNA, 21 bp, artificial sequence
  RNAi agent strand
tgaguuggcacgccuuugctt

SEQ ID NO: 108 - DNA, 21 bp, artificial sequence
  RNAi agent strand
tgaguuggcacgccuuugctt

SEQ ID NO: 109 - DNA, 23 bp, artificial sequence
  RNAi agent strand
ugugcaaaggcgugccaacucat

SEQ ID NO: 110 - DNA, 23 bp, artificial sequence
  RNAi agent strand
ugugcaaaggcgugccaacucat

SEQ ID NO: 111 - DNA, 26 bp, artificial sequence
  RNAi agent strand
tgagaagcugaggcucaaagcacuau

SEQ ID NO: 112 - DNA, 26 bp, artificial sequence
  RNAi agent strand
uauaugcuuugagccucagcuucuca

SEQ ID NO: 113 - DNA, 26 bp, artificial sequence
  RNAi agent strand
tgagaagcugaggcucaaagcauaua

SEQ ID NO: 114 - DNA, 26 bp, artificial sequence
  RNAi agent strand
uauaugcuuugagccucagcuucuca

SEQ ID NO: 115 - DNA, 26 bp, artificial sequence
  RNAi agent strand
tggucuuucacuuucuugggcucuau

SEQ ID NO: 116 - DNA, 26 bp, artificial sequence
  RNAi agent strand
uauaugcccaagaaagugaaagacca

SEQ ID NO: 117 - DNA, 26 bp, artificial sequence
  RNAi agent strand
tcacuuucuugggcuccaaacaguau

SEQ ID NO: 118 - DNA, 26 bp, artificial sequence
  RNAi agent strand
uauauguuuggagcccaagaaaguga

SEQ ID NO: 119 - DNA, 26 bp, artificial sequence
  RNAi agent strand
tucacuuucuugggcuccaaacauau

SEQ ID NO: 120 - DNA, 26 bp, artificial sequence
  RNAi agent strand
uauauuuuggagcccaagaaagugaa

SEQ ID NO: 121 - DNA, 26 bp, artificial sequence
  RNAi agent strand
tuucacuuucuugggcuccaaacuau

SEQ ID NO: 122 - DNA, 26 bp, artificial sequence
  RNAi agent strand
uauauuuggagcccaagaaagugaaa

SEQ ID NO: 123 - DNA, 26 bp, artificial sequence
  RNAi agent strand
tuuucacuuucuugggcuccaaauau

SEQ ID NO: 124 - DNA, 26 bp, artificial sequence
  RNAi agent strand
uauauuggagcccaagaaagugaaaa

SEQ ID NO: 125 - DNA, 26 bp, artificial sequence
  RNAi agent strand
tagcugaggcucaaagcacuucuuau

SEQ ID NO: 126 - DNA, 26 bp, artificial sequence
  RNAi agent strand
uauauaagugcuuugagccucagcua

SEQ ID NO: 127 - DNA, 26 bp, artificial sequence
  RNAi agent strand
tgaagcugaggcucaaagcacuuuau

SEQ ID NO: 128 - DNA, 26 bp, artificial sequence
  RNAi agent strand
uauaugugcuuugagccucagcuuca

SEQ ID NO: 129 - DNA, 26 bp, artificial sequence
  RNAi agent strand
tuuguugcggucaccacagcccguau

SEQ ID NO: 130 - DNA, 26 bp, artificial sequence
  RNAi agent strand
uauauggcuguggugaccgcaacaaa

SEQ ID NO: 131 - DNA, 26 bp, artificial sequence
  RNAi agent strand
tgcuuguugcggucaccacagccuau

SEQ ID NO: 132 - DNA, 26 bp, artificial sequence
  RNAi agent strand
uauaucuguggugaccgcaacaagca

SEQ ID NO: 133 - DNA, 26 bp, artificial sequence
  RNAi agent strand
tggcuuguugcggucaccacagcuau

SEQ ID NO: 134 - DNA, 26 bp, artificial sequence
  RNAi agent strand
uauauuguggugaccgcaacaagcca

SEQ ID NO: 135 - DNA, 26 bp, artificial sequence
  RNAi agent strand
tggucuuucacuuucuugggcucuau

SEQ ID NO: 136 - DNA, 26 bp, artificial sequence
  RNAi agent strand
uauuagcccaagaaagugaaagacca

SEQ ID NO: 137 - DNA, 26 bp, artificial sequence
  RNAi agent strand
tggucuuucacuuucuugggcucuau

SEQ ID NO: 138 - DNA, 26 bp, artificial sequence
  RNAi agent strand
uauaagcccaagaaagugaaagacca

SEQ ID NO: 139 - DNA, 26 bp, artificial sequence
  RNAi agent strand
uggucuuucacuuucuugggcucuau

SEQ ID NO: 140 - DNA, 26 bp, artificial sequence
  RNAi agent strand
uauaugcccaagaaagugaaagacca

SEQ ID NO: 141 - DNA, 26 bp, artificial sequence
  RNAi agent strand
uggucuuucacuuucuugggcucuau

SEQ ID NO: 142 - DNA, 26 bp, artificial sequence
  RNAi agent strand
uuagagcccaagaaagugaaagacca

SEQ ID NO: 143 - DNA, 26 bp, artificial sequence
  RNAi agent strand
uggucuuucacuuucuugggcucuau

SEQ ID NO: 144 - DNA, 26 bp, artificial sequence
  RNAi agent strand
uuauugcccaagaaagugaaagacca

SEQ ID NO: 145 - DNA, 26 bp, artificial sequence
  RNAi agent strand
uggucuuucacuuucuugggcucuau

SEQ ID NO: 146 - DNA, 26 bp, artificial sequence
  RNAi agent strand
uugaugcccaagaaagugaaagacca

SEQ ID NO: 147 - DNA, 26 bp, artificial sequence
  RNAi agent strand
uggucuuucacuuucuugggcucuau

SEQ ID NO: 148 - DNA, 26 bp, artificial sequence
  RNAi agent strand
uaugagcccaagaaagugaaagacca

SEQ ID NO: 149 - DNA, 26 bp, artificial sequence
  RNAi agent strand
tggucuuucacuuucuugggcucuau

SEQ ID NO: 150 - DNA, 26 bp, artificial sequence
  RNAi agent strand
auagagcccaagaaagugaaagacca

SEQ ID NO: 151 - DNA, 26 bp, artificial sequence
  RNAi agent strand
ugaagcugaggcucaaagcacuuuau

SEQ ID NO: 152 - DNA, 26 bp, artificial sequence
  RNAi agent strand
uauaugugcuuugagccucagcuuca

SEQ ID NO: 153 - DNA, 26 bp, artificial sequence
  RNAi agent strand
tgaagcugaggcucaaagcacuuuau

SEQ ID NO: 154 - DNA, 26 bp, artificial sequence
  RNAi agent strand
uauaugugcuuugagccucagcuuca

SEQ ID NO: 155 - DNA, 26 bp, artificial sequence
  RNAi agent strand
uggucuuucacuuucuugggcucuau

SEQ ID NO: 156 - DNA, 26 bp, artificial sequence
  RNAi agent strand
uauaugcccaagaaagugaaagacca

SEQ ID NO: 157 - DNA, 26 bp, artificial sequence
  RNAi agent strand
uggucuuucacuuucuugggctcuau

SEQ ID NO: 158 - DNA, 26 bp, artificial sequence
  RNAi agent strand
uauaugcccaagaaagugaaagacca

SEQ ID NO: 159 - DNA, 26 bp, artificial sequence
  RNAi agent strand
uggucuuucacuuucuugggcucuau

SEQ ID NO: 160 - DNA, 26 bp, artificial sequence
  RNAi agent strand
uauaugcccaagaaagugaaagauau

SEQ ID NO: 161 - DNA, 26 bp, artificial sequence
  RNAi agent strand
uauggucuuucacuuucuugggcucu

SEQ ID NO: 162 - DNA, 26 bp, artificial sequence
  RNAi agent strand
uaugcccaagaaagugaaagaccuau

SEQ ID NO: 163 - DNA, 26 bp, artificial sequence
  RNAi agent strand
uauggucuuucacuuucuugggcucu

SEQ ID NO: 164 - DNA, 26 bp, artificial sequence
  RNAi agent strand
uaugcccaagaaagugaaagaccaau

SEQ ID NO: 165 - DNA, 26 bp, artificial sequence
  RNAi agent strand
uauggucuuucacuuucuugggcucu

SEQ ID NO: 166 - DNA, 26 bp, artificial sequence
  RNAi agent strand
uaugcccaagaaagugaaagaccauu

SEQ ID NO: 167 - DNA, 26 bp, artificial sequence
  RNAi agent strand
uauggucuuucacuuucuugggcucu

SEQ ID NO: 168 - DNA, 26 bp, artificial sequence
  RNAi agent strand
uaugcccaagaaagugaaagaccaua

SEQ ID NO: 169 - DNA, 26 bp, artificial sequence
  RNAi agent strand
uauggucuuucacuuucuugggcucu

SEQ ID NO: 170 - DNA, 26 bp, artificial sequence
  RNAi agent strand
uaugcccaagaaagugaaagaccuau

SEQ ID NO: 171 - DNA, 21 bp, artificial sequence
  RNAi agent strand
tgaguuggcacgccuuugctt

SEQ ID NO: 172 - DNA, 23 bp, artificial sequence
  RNAi agent strand
ugugcaaaggcgugccaacucat

SEQ ID NO: 173 - DNA, 21 bp, artificial sequence
  RNAi agent strand
tgaguuggcacgccuuugctt

SEQ ID NO: 174 - DNA, 23 bp, artificial sequence
  RNAi agent strand
ugugcaaaggcgugccaacucat

SEQ ID NO: 175 - DNA, 26 bp, artificial sequence
  RNAi agent strand
tgacaccugauucuguuucugaguau

SEQ ID NO: 176 - DNA, 26 bp, artificial sequence
  RNAi agent strand
uauaucagaaacagaaucagguguca

SEQ ID NO: 177 - DNA, 26 bp, artificial sequence
  RNAi agent strand
tgagaaugagccucgauaacucuuau

SEQ ID NO: 178 - DNA, 26 bp, artificial sequence
  RNAi agent strand
uauauaguuaucgaggcucauucuca

SEQ ID NO: 179 - DNA, 26 bp, artificial sequence
  RNAi agent strand
tgagaaugagccucgauaacucuuau

SEQ ID NO: 180 - DNA, 26 bp, artificial sequence
  RNAi agent strand
uauauaguuaucgaggcucauucuca

SEQ ID NO: 181 - DNA, 26 bp, artificial sequence
  RNAi agent strand
tgcgucugagcauugugucagguuau

SEQ ID NO: 182 - DNA, 26 bp, artificial sequence
  RNAi agent strand
uauaucugacacaaugcucagacgca

SEQ ID NO: 183 - DNA, 26 bp, artificial sequence
  RNAi agent strand
tugcgucugagcauugugucagguau

SEQ ID NO: 184 - DNA, 26 bp, artificial sequence
  RNAi agent strand
uauauugacacaaugcucagacgcaa

SEQ ID NO: 185 - DNA, 26 bp, artificial sequence
  RNAi agent strand
taagggcgaaucucagcaucugguau

SEQ ID NO: 186 - DNA, 26 bp, artificial sequence
  RNAi agent strand
uauauagaugcugagauucgcccuua

SEQ ID NO: 187 - DNA, 26 bp, artificial sequence
  RNAi agent strand
tgagaaugagccucgauaacucuuau

SEQ ID NO: 188 - DNA, 26 bp, artificial sequence
  RNAi agent strand
uauauaguuaucgaggcucauucuca

SEQ ID NO: 189 - DNA, 26 bp, artificial sequence
  RNAi agent strand
ugagaaugagccucgauaacuctuau

SEQ ID NO: 190 - DNA, 26 bp, artificial sequence
  RNAi agent strand
uauauaguuaucgaggcucauucuca

SEQ ID NO: 191 - DNA, 26 bp, artificial sequence
  RNAi agent strand
ugagaaugagccucgauaacucuuau

SEQ ID NO: 192 - DNA, 26 bp, artificial sequence
  RNAi agent strand
uauauaguuaucgaggcucauucuca

SEQ ID NO: 193 - DNA, 26 bp, artificial sequence
  RNAi agent strand
ugagaaugagccucgauaacucuuau

SEQ ID NO: 194 - DNA, 26 bp, artificial sequence
  RNAi agent strand
uauauaguuaucgaggcucauucuca

SEQ ID NO: 195 - DNA, 26 bp, artificial sequence
  RNAi agent strand
ugagaaugagccucgauaacucuuau

SEQ ID NO: 196 - DNA, 26 bp, artificial sequence
  RNAi agent strand
uauauaguuaucgaggcucauucuca

SEQ ID NO: 197 - DNA, 26 bp, artificial sequence
  RNAi agent strand
tgacaccugauucuguuucugaguau

SEQ ID NO: 198 - DNA, 26 bp, artificial sequence
  RNAi agent strand
uauaucagaaacagaaucagguguca

SEQ ID NO: 199 - DNA, 26 bp, artificial sequence
  RNAi agent strand
ugacaccugauucuguuucugaguau

SEQ ID NO: 200 - DNA, 26 bp, artificial sequence
  RNAi agent strand
uauaucagaaacagaaucagguguca

SEQ ID NO: 201 - DNA, 26 bp, artificial sequence
  RNAi agent strand
ugacaccugauucuguuucugaguau

SEQ ID NO: 202 - DNA, 26 bp, artificial sequence
  RNAi agent strand
uauaucagaaacagaaucagguguca

SEQ ID NO: 203 - DNA, 26 bp, artificial sequence
  RNAi agent strand
ugacaccugauucuguuucugaguau

SEQ ID NO: 204 - DNA, 26 bp, artificial sequence
  RNAi agent strand
uauaucagaaacagaaucagguguca

SEQ ID NO: 205 - DNA, 26 bp, artificial sequence
  RNAi agent strand
tcguauaacaauaaggggcugccuau

SEQ ID NO: 206 - DNA, 26 bp, artificial sequence
  RNAi agent strand
uauaucagccccuuauuguuauacga

SEQ ID NO: 207 - DNA, 26 bp, artificial sequence
  RNAi agent strand
ucguauaacaauaaggggcugccuau

SEQ ID NO: 208 - DNA, 26 bp, artificial sequence
  RNAi agent strand
uauaucagccccuuauuguuauacga

SEQ ID NO: 209 - DNA, 26 bp, artificial sequence
  RNAi agent strand
ucguauaacaauaaggggcugccuau

SEQ ID NO: 210 - DNA, 26 bp, artificial sequence
  RNAi agent strand
uauaucagccccuuauuguuauacga

SEQ ID NO: 211 - DNA, 26 bp, artificial sequence
  RNAi agent strand
tuucaugaaaucguuacguuggcugu

SEQ ID NO: 212 - DNA, 26 bp, artificial sequence
  RNAi agent strand
uauaucaacguaacgauuucaugaaa

SEQ ID NO: 213 - DNA, 26 bp, artificial sequence
  RNAi agent strand
tuucaugaaaucguuacgucggcugu

SEQ ID NO: 214 - DNA, 26 bp, artificial sequence
  RNAi agent strand
uauaucgacguaacgauuucaugaaa

SEQ ID NO: 215 - DNA, 26 bp, artificial sequence
  RNAi agent strand
tuucaugaaaucguuacguuggcuau

SEQ ID NO: 216 - DNA, 26 bp, artificial sequence
  RNAi agent strand
uauaucaacguaacgauuucaugaaa

SEQ ID NO: 217 - DNA, 26 bp, artificial sequence
  RNAi agent strand
tuucaugaaaucguuacguuggcutt

SEQ ID NO: 218 - DNA, 26 bp, artificial sequence
  RNAi agent strand
uauaucaacguaacgauuucaugaaa

SEQ ID NO: 219 - DNA, 26 bp, artificial sequence
  RNAi agent strand
tuucaugaaaucguuacgucggcuau

SEQ ID NO: 220 - DNA, 26 bp, artificial sequence
  RNAi agent strand
uauaucgacguaacgauuucaugaaa